(19) United States
(12) Patent Application Publication
(10) Pub. No.: US 2023/0302133 A1
(43) Pub. Date: Sep. 28, 2023

(54) TARGETED PROTEIN DEGRADATION IN THERAPEUTIC CELLS

(71) Applicant: The Regents of the University of California, Oakland, CA (US)

(72) Inventors: Andrew H. Ng, San Francisco, CA (US); Matthew Kim, San Francisco, CA (US)

(21) Appl. No.: 18/020,611

(22) PCT Filed: Aug. 24, 2021
(86) PCT No.: PCT/US2021/047391

§ 371 Date: Feb. 9, 2023

Related U.S. Application Data
(60) Provisional application No. 63/070,166, filed on Aug. 25, 2020.

(51) Int. Cl. A61K 39/00; A61K 38/17; C12N 5/0783

(52) U.S. Cl. CPC A61K 39/4631; A61K 39/4611; A61K 39/46433; A61K 38/1709; C12N 5/0636

(57) ABSTRACT

Described herein is a therapeutic cell that expresses a fusion protein comprising: (a) a target-binding domain; and (b) a degradation domain, e.g., a degron or E3 ligase-recruiting domain, that is heterologous to the target-binding domain. In the therapeutic cell, binding of the fusion protein to a target protein via the target-binding domain induces degradation of the target protein. The therapeutic cell can be an immunostimulatory cell, an immunoinhibitory cell or a stem cell, for example. Methods of treatment using the cell are also provided.

### CROSS-REFERENCING

This application claims the benefit of provisional application Ser. No. 63/070,166, filed on Aug. 25, 2020, which application is incorporated by reference herein for all purposes.

### GOVERNMENT RIGHTS

This invention was made with government support under grant no. HR0011-16-2-0045 awarded by Defense Advanced Research Projects Agency. The government has certain rights in the invention.

### BACKGROUND

Regulating the activity of specific proteins inside a cell is a central challenge to cell engineering. Existing methods largely focus on regulating gene expression. However, even with new genome engineering technologies, it can be difficult to challenging to control the activity of an endogenous gene. Methods for fully synthetic transcriptional regulation are limited. This disclosure provides a new solution to this problem.

### SUMMARY

Provided herein is a therapeutic cell that expresses a fusion protein comprising: (a) a target-binding domain; and (b) a degradation domain that is heterologous to the target-binding domain. The degradation domain can be a degron or an E3 ligase-recruiting domain, for example. In this cell, binding of the fusion protein to a target protein via the target-binding domain induces degradation of the target protein. For example, the degradation domain can be a degron, a domain that directly interacts with the E3 ligase, or domain that indirectly interacts with the E3 ligase.

The target-binding domain can be a scFv, nanobody or a non-antibody target-binding domain such as a synthetic leucine zipper, SH2 domain, SH3 domain, or PDZ domain, etc. In some embodiments, the target-binding domain may bind to a motif having a post-translational modification. In these embodiments, the target-binding domain may comprise an SH2 domain or PTB domain (which bind to motifs that have a phosphotyrosine), a FHA, or WD40-repeat domain (which can bind to motifs that contain phosphoserine or phosphothreonine), a bromo domain (which bind to motifs that have an acylated lysine) or a chromo domain (which bind to motifs that have an methylated lysine). These latter embodiments allow one to degrade signaling proteins, but only when they are in the process of signaling.

The target protein can be endogenous to the cell, or exogenous to the cell and, in some embodiments, binding of the fusion protein to the target protein may be chemically inducible.

In any embodiment, the target-binding domain may be C-terminal or N-terminal to the degradation domain in the fusion protein.

These and other advantages may be become apparent in view of the following discussion.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates an example of the present fusion protein.

FIG. 2 schematically illustrates various models for cullin-RING E3 ligases. These complexes promote the transfer of ubiquitin from the E2 to the substrate, which targets the protein for degradation. Many complexes contain an adapter protein (e.g., SKP1 for CUL1 and CUL7, Elongin B/C for CUL2 and CUL5, BTB for CUL3 and DDB1 for CUL4A/b) as well as a receptor protein (F-box proteins for CUL1, VHL-box proteins for CUL2, DCAFs for CUL4A and 4B, SOCS for CUL5 and FbxW8 for CUL7) and a RING protein (RB1/2).

FIG. 3: Lysine to arginine substitution significantly improves STUD activity. Either a GFP nanobody (vhhGFP4) or SynZIP (SZ18) were used to target a GFP (or in the case of the SZ18 STUD, GFP-SZ17. SZ17 and SZ18 form a cognate pair). GFP % Degradation was measured compared to GFP fluorescence in the absence of the STUD.

FIG. 4: MG132 proteasome inhibitor confirms the effect of STUD is mediated by the proteasome. Primary human CD4+T cells expressing different variants of the GFP nanobody STUD were few 5 uM MG132 and fluorescence was measured at 1 and 3 hours post induction. The mutant nanobody was the only experimental group that exhibited an increase in fluorescence over time, suggesting the effect of the STUD is mediated by protein degradation through the proteasome.

FIG. 5: Optimizing STUD activity via linker modification in Jurkat cells. A variety of flexible (GS) and rigid linkers were tested between the SynZIP targeting domain and degron on the STUD. We observed that flexible linkers generally outperformed rigid linkers, and in particular the 5xGS linker produced the greatest degradation

FIG. 6: Design of a circuit to test STUD induced degradation of a synthetic transcription factor. VPR-NS3-ZF3 drives activation of the pZF3(8×)_ybTATA promoter in response to induction with GRZ. Three different circuit configurations were explored. Feedback, where STUD is driven off the pZF3 promoter, GFP alone, where no STUD is expressed, and Constitutive STUD, where the STUD is expressed off the pPGK promoter

FIG. 7: ZF3 circuit dose responses demonstrate the functionality of the soluble STUD to degrade a transcription factor. The circuits shown in FIG. 3 were transduced into Jurkat cells and induced with a range of GRZ concentrations to activate the TF. GFP fluorescence was measured 72 hours later

FIG. 8: Testing the ability of soluble STUDs to target a CAR-SZ17 fusion for degradation in Jurkat cells. Four different linker lengths between the SynZIP18 on the STUD and degron were tested. A control where the degron was directly fused to the CAR generated the most degradation.

FIG. 9: Design of membrane targeting STUD. DAP10 extracellular domain (ECD) contains a signal sequence that traffics the protein in the membrane. The CD8 transmembrane domain (TMD) embeds in membrane and is linked to the soluble STUD via a linker.

FIG. 10: Degradation of CAR in primary human CD4+T cells. Rigid15 linker between CD8 TMD and soluble STUD mediated the greatest amount of CAR degradation as measured by staining for the myc-tag present on the CAR and flow cytometry.

FIG. 11: Degradation of SynNotch in primary human CD4+T cells. Rigid15 linker between CD8 TMD and soluble STUD mediated the greatest amount of SynNotch degradation as measured by staining for the myc-tag present on the SynNotch and flow cytometry.

FIG. 12. Overview and demonstration of STUD system. (A) Left: Cartoon depiction of truncated ubiquitin proteasome pathway (UPP). Right: Cartoon of example of Synthetic Targeter of Ubiquitination and Degradation (“STUD”) bridging a target protein of interest with the endogenous UPP to initiate degradation of the target. (B) Top: Cartoon depiction of plasmids used in demonstration of STUD-induced degradation of green fluorescent protein (GFP) target in Jurkat T cells. Jurkat cells were lentivirally transduced with two plasmids. The first encodes a STUD, or control with a mutated degron, and a mCherry transduction marker separated by a 2A element and the second encodes GFP target protein and a BFP transduction marker separated by a 2A element. In the case of the SynZip STUD, the GFP target is fused to a heterodimeric SynZip protein complementary to the binding domain on the STUD. These transduced cells are analyzed for fluorescence by flow cytometry 48 hours after removal of virus. Cells are first gated on these transduction markers to isolate relevant populations. Then, normalized GFP fluorescence is calculated by normalizing each cell's GFP fluorescence, as obtained by flow cytometry, by its BFP fluorescence. Then, the median normalized GFP fluorescence of each flow cytometry distribution is calculated and shown in the bar plot. Each dot represents the mean of the median normalized GFP fluorescence of three technical replicates in three independent experiments. The flow cytometry distributions of one of these technical replicates is shown below. In the distributions, the “+TRGN” (SEQ ID NO:50) condition corresponds to the “RRRG−” (SEQ ID NO:32) condition in the bar plot. Error bars represent the standard deviation. (C) STUD degradation of a GFP target in various mammalian cell lines. Median normalized GFP values are calculated as in (B), but each dot here represents a technical replicate. Error bars represent standard deviation.

FIG. 13. STUD degradation of GFP is mediated by the cullins in UPP. Representative flow cytometry distributions of GFP fluorescence of Jurkat T cells expressing the SynZip STUD described in FIG. 12B treated with one of three drugs or a DMSO vehicle control. Distributions are representative of three independent experiments.

FIG. 14. Tethering of STUD to plasma membrane allows for functional knockdown for second-generation chimeric antigen receptors (CAR). (A) Cartoon diagram of membrane tethered STUD (‘memSTUD’) and non-functional (‘NF’) control relative to original ‘soluble’ STUD design. The DNA cartoon represents the plasmid used in experiments done in this panel. (RRRG (SEQ ID NO:32); TRGN (SEQ ID NO:50)) (B) Plasmid diagramed in FIG. 14A transduced into Jurkat T cells and CAR fluorescence measured 72 hours removal of lentivirus. CAR fluorescence is measured by antibody stain for myc tag fused to CAR extracellular domain. Bar plot displays the median fluorescence based on antibody stain signal and representative flow cytometry distributions show. Dots represent three independent experiments and errors bars show standard deviation. (C) Plasmid in (A) is transduced into CD8+primary T cells and anti-HER2 4-1BB CD3zeta CAR expression and activation is assayed. Left: Representative flow cytometry distributions of anti-HER2 4-1BB CD3zeta CAR fluorescence by antibody stain. Middle: Engineered T cells are cocultured with K562 target cells expressing various levels of HER2 antigen for 72 hours and lysis is measured by flow cytometry. Lysis is calculated relative to lysis observed when UnT cells are cocultured with target cells. Right: Median fluorescence of T cell activation marker CD25 after coculture is measured by antibody stain for CD25 and flow cytometry. (D) Plasmid in (A) is transduced into CD8+primary T cells and anti-CD19 4-1BB CD3zeta CAR expression and activation is assayed. Left: Cullin inhibitor MLN4924 is added to engineered T cells to rescue degradation of CAR by STUD. Bar plot shows CAR fluorescence by antibody stain and flow cytometry after 5 hours of incubation with inhibitor. Black bars represent each condition with DMSO vehicle control. Middle: Engineered T cells are cocultured with NALM6 target cells for 72 hours and lysis is measured by flow cytometry. Lysis is calculated relative to lysis of NALM6 cells cultured with UnT T cells. Right: Median fluorescence of T cell activation marker CD25 after coculture is measured by antibody stain for CD25 and flow cytometry.

FIG. 15. Design of new synthetic receptor allows for antigen triggered degradation of cytosolic proteins. (A) Cartoon diagram of novel NotchSTUD synthetic receptor that couples antigen binding to target degradation. (B) Left: Representative flow cytometry distribution of target GFP fluorescence of cells gated on mCherry and tagBFP co-transduction markers 72 hours after coculture. Right: Quantification of median GFP target fluorescence normalized by tagBFP co-transduction marker following 72 hours of coculture with HER2+K562 target cells or WT K562 target cells. Black bars on bar plot represent standard deviation of three technical triplicates.

FIG. 16. STUDs can be composed into negative feedback circuit to regulate synthetic transcription factor (SynTF). (A) Cartoon diagram of plasmids used in negative feedback circuit transduced into Jurkat T cells. Synthetic transcription factors used in this work (grey) are made up of a transcriptional activation domain (AD) and a DNA binding domain (DBD) separated by a NS3 protease. In the absence of the NS3 inhibitor grazoprevir (GZV), the TF is destabilized and non-functional. On the other hand, the addition of GZV stabilizes the TF and allows for transcription of the gene cassette downstream of the SynTF promoter (pSynTF). Three types of circuits were designed: (1) A negative feedback circuit that has the SynTF driving expression of the GFP reporter and the STUD which results in degradation of the SynTF and shut off of the circuit. (2) an open loop control where there is no STUD present to gauge the maximum activity of the SynTF and (3) an open loop control where the STUD is constitutively present and degrading the SynTF. (B) Dose response of circuit after 72 hours of incubation with GZV at 37 C. The plot displays GZV concentration versus Fold change of the median GFP relative to the DMSO vehicle control.

FIG. 17. Dose dependent degradation of STUDs allow for use as ON switch CAR. We engineer CD8+primary human T cells that express an antiCD19BBz CAR fused to a SynZip and a STUD to recognizes the SynZip. As a control, the CD8+primary human T cells that express the same CAR without the SynZip and the SynZip STUD were also engineered. Both these cell lines were cocultured and an untransduced (UnT) control with NALM6 target cells for 72 hours at 37 C in the presence of various concentrations of MLN4924. Lysis of target cells was calculated by each line relative to NALM6 cells cultured alone by flow cytometry.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab-SH is the designation herein for Fab in which the cysteine residue(s) of the constant domains bear a free thiol group. F(ab)2 antibody fragments originally were produced as pairs of Fab fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. In some cases, the first member of a specific binding pair present in the extracellular domain of a chimeric Notch receptor polypeptide of the present disclosure binds specifically to a second member of the specific binding pair. “Specific binding” refers to binding with an affinity of at least about 10-7 M or greater, e.g., 5×10-7 M, 10-8 M, 5×10-8 M, and greater. “Non-specific binding” refers to binding with an affinity of less than about 10-7 M, e.g., binding with an affinity of 10-6 M, 10-5 M, 10-4 M, etc.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As noted above, disclosed herein is a therapeutic cell (e.g., a recombinant immune cell such as a CAR T, a Treg cell or stem cell) that expresses a fusion protein (i.e., contains an expression cassette comprising a promoter and, operably linked to the promoter, a coding sequence that encodes the fusion protein), where the fusion protein comprises: (a) a target-binding domain; and (b) a degradation domain that is heterologous to the target-binding domain, where the degradation domain may a degron or E3 ligase-recruiting domain.

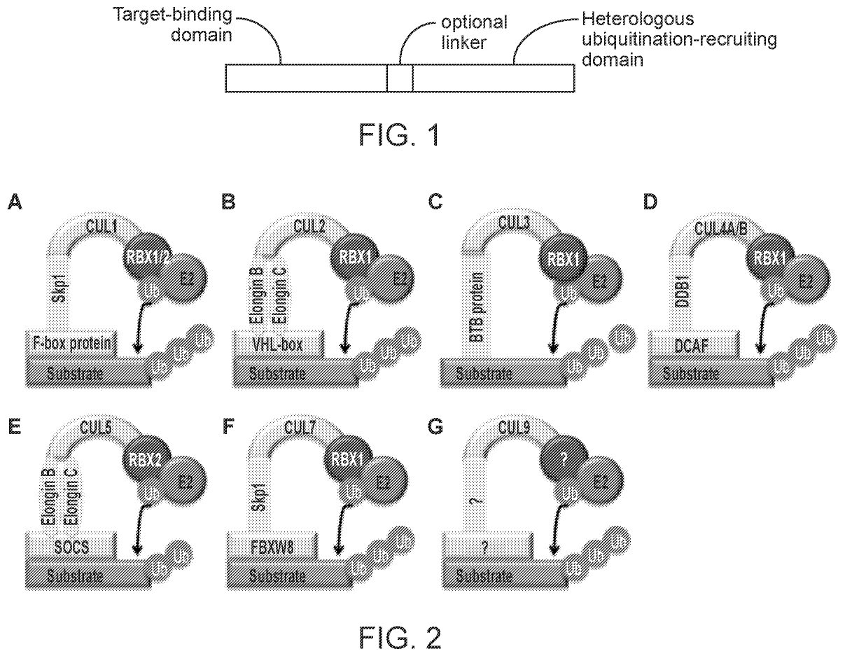

This fusion protein is schematically illustrated in FIG. 1. The target-binding domain can be N-terminal or C-terminal to the degradation domain, and, as shown, the fusion protein may optionally contain a linker between the target-binding domain and the degradation domain. In the therapeutic cell, binding of the fusion protein to a target protein via the target-binding domain induces degradation of the target protein. Degradation may be ubiquitination-mediated or not ubiquitination-mediated, depending on which degradation domain is used. Various degradation domains are described below.

Degrons

Degrons are relatively short (typically under 100 amino acids) sequences that, when they are present in a protein, target that protein for degradation. Degrons include ubiquitin-dependent degrons and ubiquitin-independent degrons. Examples of degrons include ubiquitin (which is approximately 76 amino acids in length), PEST sequences (which are approximately 10 to 60 amino acids in length and are, rich in P (proline), E (glutamate), S (serine). and T (threonine)), N-degrons (which are short N-terminal sequences), C degrons (which are short N-terminal sequences), unstructured initiation sites and short sequences rich in acceptor lysines. Degrons are diverse in sequence and have been extensively reviewed (see. e.g., Varshavsky, Proc. Natl. Acad. Sci. 2019 116: 358-366, Varshavsky, Protein Sci. 2011 20: 1298-1.345; Natsume et al., Annu Rev. Genet 2017 51: 83-102; Rechsteiner et al., Trends Biochem Sci. 1996 21: 267-271; Herbst. et al., Oncogene 2004 23: 3863-3871; Prakash, Nat. Struct. Mol. Biol. 2004 11: 830-837; Guharoy et al., Nat. Commun. 2016 7: 10239 and Chassin et al. Nature Comm. 2019 10).

Examples of C-degrons suitable for use in a fusion protein are listed below (see Koren et al., Cell 2018 173: 1622-1635):

| Name | Sequence | ID NO | Motif |
|---|---|---|---|
| FRA68_EMID1 | RGKRGGHATNYRIVAPRSRDERG* | 1 | RG* |
| FRA69_CHGA | ESLSAIEAELEKVAHQLQALRRG* | 2 | RG* |
| FRA70_MAGEA3 | KISGGPHISYPPLHEWVLREGEE* | 3 | EE* |
| FRA71_MAGEA3EE | KISGGPHISYPPLHEWVLREGAA* | 4 | EE* to Ax/A* |
| toAA | | | |
| FRA72_PIK3C2B | LRELDLAQEKTGWFALGSRSHGTL* | 5 | RxxGxx* |
| FRA73_PXN | LRELDLAQEKTGWFALGSRHCGRT* | 6 | RxxGxx* |
| FRA74_Peptide35 | YKKAGSGIPLRMNSLFRKRNKGK* | 7 | RxxGxx* |
| FRA75_CDK5R1 | VFSDLKNESGQEDKKRLLLGLDR* | 8 | R* motif, |
| FRA76_CDK5R1trunc | VFSDLKNESGQEDKKRLLLGLD* | 9 | R truncated, |
| FRA77_SIL1 | DGEDEGYFQELLGSVNSLLKELR* | 10 | R* |
| fRA78_SIL1trunc | DGEDEGYFQELLGSVNSLLKEL* | 11 | R truncated, |
| fRA79_N-Myc | LEKEKLQARQQQLLKKIEHARTC* | 12 | Rxx* |
| FRA80_N- | LEKEKLQARQQQLLKKIEHA* | 13 | Rxx* to A*, |
| Myctrunc | | | |
| FRA81_MSRB2 | GPGPNGQRFCINSAALKFKPRKH* | 14 | Rxx* |
| FRA82_OR4C13 | LRNAQMKNAIRKLCSRKAISSVK* | 15 | Vx* motif |
| FRA83_OR4C13Dmut | LRNAQMKNAIRKLCSRKAISSDK* | 16 | Vx to Dx |
| FRA84_SREBF2 | RRSCNDCQQMIVKLGGGTAIAAS* | 17 | Ax* |
| FRA85_SREBF2- | RRSCNDCQQMIVKLGGGTAIADS* | 18 | Ax |
| Dmut | | | |
| FRA86_CPS1 | QKSRKVDSKSLFHYRQYSAGKAA* | 19 | AA* |
| FRA87_CPS1DDmut | QKSRKVDSKSLFHYRQYSAGKDD* | 20 | AA to DD, |
| FRA88_CPS1CText | QKSRKVDSKSLFHYRQYSAGKAAKASTN* | 21 | AA Ct |
| FRA89_EPHB2 | REIQGIFFKEDSHKESNDCSCGG* | 22 | GG |
| FRA90_PDGFC | SLTDVALEHHEECDCVCRGSTGG* | 23 | GG |
| FRA91_ASCC3 | RRLDGKEEDEKMSRASDRFRGLR* | 24 | RG/R* dual |
| RA2102_degBon1 | TRGVEEVAEGVVLLRRRGN* | 25 | Rxx*/RxxG |
| RA2106_Clone1 | (GIPLR)NLGIR* | 26 | RG/R* dual |
| RA2107_Clone6 | (GIPLR)QRKLQRTSRG* | 27 | RG* |
| RA2108_Clone6G | (GIPLR)QRKLQRTSRA* | 28 | RG* to A*, |
| toA | | | |
| RA2109_Clone8 | (GIPLR)PHKRLLKGSQYG* | 29 | RG*- like |

Further examples of C-degrons suitable for use in a fusion protein are listed below (see Bortger, Nat. Chem Biol 7.531-537):

| | | |
|---|---|---|
| RA2103_degBon2 | TRGVEEVAEGVVLLRRRG* | dual motif (RG*) |
| | (SEQ ID NO: 30) | |
| RA2104_degBon3 | TRRRGN* (SEQ ID | stronger variant |
| | NO: 31) | |
| RA2105_degBon4 | RRRG* (SEQ ID | strongest variant |
| | NO: 32) | |

One example of an N-degron suitable for use hi a fusion protein is listed below (see Bachmair et al, Cell 1989 56, 1019-1032). This sequence is a fusion of Ubiquitin and N terminus of B-gal.

| | |
|---|---|
| Ubi-R | QIFVKTLTGKTITLEVESSDTIDNVKSKIQDKEGIPPDQQ |
| | RLIFAGKQLEDGRTLSDYNIQKESTLHLVLRLRGGRHGSG |
| | AWLLPVSLVRRRTTLAPNTQTASPRALADSLMQRS |
| | (SEQ ID NO: 33) |

One example of a PEST sequence suitable for use in a fusion protein is listed below (see Rogers et a; Science 1986 234: 364-8). There are many examples of PEST sequences.

| Name | Sequence | Origin |
|---|---|---|
| p53 PEST | DDLLLPQDVEEFFEGPSEALR | p53 |
| | (SEQ ID NO: 34) | |

Further examples of degrons that could be employed are shown below. These sequences are disclosed in Hon et al. (Nature, 2002 417: 975-8), Fan et al. (Nat. Neurosci. 2014 17: 471-480), Gu et al. (Molecular and Cellular Biology 2000 20: 1243-1253), Melvin et al. (Analyst 2016 141:570-8) and Zhang et al. (Developmental Cell 2019 48: 329-344).

| Name | Sequence | ID NO | Origin | E3 Ligase |
|---|---|---|---|---|
| VHLdeg | ALAPYIP | 35 | HIF-1a | VHL |
| CMAdeg | KFERQKILDQRFFE | 36 | RNaseA-hsc70- | Lysosome |
| | | | hemoglobin | |
| MDM2deg | PLSSSVPSQKTYQGSYGFRLG | 37 | p54(92-112) | MDM2 |
| MDM2(short)deg | GSYG | 38 | p54(92-112) | MDM2 |
| SPOP(2)deg | DVQKADVSST | 39 | SRC3 | SPOP |
| SPOP(3)deg | SPDSSTSP | 40 | Nanog | SPOP |
| BONGERdeg | RRRG | 32 | Synthetic | Unknown |
| iNOSdeg | DINNN | 41 | iNOS | Unknown |

In this fusion protein, the degron works in trans, meaning that the target protein that is degraded is a different protein, i.e., the protein that the fusion protein (which contains the degron) binds to.

E3 Ligase Recruiting Domains

In the cell, the target-binding domain of the fusion protein binds to a target protein and recruits it into an E3-ligase complex, thereby causing the target to be ubiquitinated and degraded. In some embodiments, the E3 ligase recruiting domain of the fusion protein may interact with an E3 ligase directly or indirectly. In these embodiments, the E3 ligase is endogenous to the cell. FIG. 2 illustrates some of the current models of how substrates are recruited for degradation. As shown in panels A, B, D, E and F many complexes contain an adapter protein (e.g., Skp1, Elongin B/C or DDB1) that links the E3 ligase (a cullin) to a protein that binds to the substrate. The protein that binds to the substrate is referred to as a “receptor” (an may be an F-box protein, VHL-box protein, DCAF, SOCS, for example). In one model (c), the receptor binds directly to the E3 ligase. The degradation domain of a fusion protein can contain any of the interaction domains shown in FIG. 2 (e.g., the E3 ligase interaction domain of an adapter protein or receptor, or the adapter protein-interaction domain of a receptor). As would be apparent, if the fusion protein contains the E3 ligase interaction domain of an adapter protein or receptor, or the adapter protein-interaction domain of a receptor, then the fusion protein does not need to contain other parts of the protein. For example, if the target binding domain of the fusion protein is from an adapter protein, then the fusion protein does not need to contain the part of the adapter protein that binds to the receptor. In these embodiments, the fusion protein may contain the E3 ligase binding domain of an adapter protein but not the receptor binding domain of the adapter protein. Likewise, if the target binding domain of the fusion protein is from a receptor protein, then the fusion protein does not need to contain the part of the receptor protein that binds to the endogenous substrate. In these embodiments, the fusion protein may contain the adapter protein binding domain of a receptor but not the substrate binding domain of the receptor.

In some embodiments, the E3 ligase recruiting domain can directly interact with Cullin protein. Examples of E3 ligase recruiting domains that directly interact with a Cullin protein may be found in E3 complex adapter proteins and in some substrate receptors (e.g., BTB, as shown in FIG. 2).

For example, an E3 ligase recruiting domain that directly interacts with an E3 ligase may have the Cullin binding region of an adapter protein, such as Skp1, ElonginB/C, or DDB1 (as illustrated in FIG. 2). These Cullin binding regions have been studied in depth (see, e.g., Schulman Nature 2000 408: 381-386, Zheng et al. Nature 2002 416: 703 and Fischer Nature 2014 512: 49-53) and the sequence of these domains can be readily derived from these studies. For example, Skp1 and ElonginC have a conserved BTB/POZ domain that interacts with CUL1 and CUL2/5, respectively.

In another example, an E3 ligase recruiting domain that directly interacts with a Cullin protein may have a BTB domain. Examples of BTB domains can be found in substrate receptors that interact directly with CUL3. Examples of such substrate receptors that directly interact with CUL3 include SPOP and KLHL family (e.g., Keap1) members.

These Cullin binding regions have been studied in depth (see, e.g., Stogios et al. Genome Biology 2005 6: R82, Zhuang et al. Molecular Cell 2009 36: 39-50 and Lee et al. Molecular Cell 2009 36: 131-140) and the sequence of these domain can be readily derived from these studies.

In other embodiments, the E3 ligase recruiting domain may indirectly interact with an E3 ligase protein. This interaction may be via an adapter protein. Examples of E3 ligase recruiting domains that indirectly interact with an E3 ligase may be found in some E3 substrate receptors (e.g., those receptors that interact with a Cullin via an adapter protein).

For example, an E3 ligase recruiting domain that indirectly interacts with an E3 ligase may have an F-box. Examples of F-box domains can be found in E3 substrate receptors that interact with Cullin-1 or Cullin-7 via Skp1. Canonical F-box proteins that bind Skp1 include FBW1A (beta-TRCP), Skp2, and Fbw7. The F box has been studied in depth (Su et al. Proc. Natl. Acad. Sci. 2003 100: 12729-12734; Schulman, Nature 2000 408:381-386, Yumimoto Journal of Biological Chemistry 2-13 288: 28488-28502 and Skaar, Nature Reviews Molecular Cell Biology 2013 14: 369-381) and the sequence of this domain can be readily derived from these studies.

In another example, an E3 ligase recruiting domain may have a VHL- or SOCS-box. Examples of VHL- and SOCS-box domains can be found in E3 substrate receptors that interact Cullin-2 or Cullin-7 via Elongin B/C. Examples of F-box domains include members of suppressors of cytokine signaling (SOCS) family of proteins (e.g., Socs1, Socs3) as well as pVHL. The structure of these domains has been studied in depth (see. e.g., Liau et al. Nature Comm 2018 9: 1558, Stebbins et al. Science 1999 284: 455-461, Kamura, Genes & Development 2003 18: 3055-3065 and Linossi IUBMB Life 2012 64: 316-323) and the sequence of this domain can be readily derived from these studies.

In another example, an E3 ligase recruiting domain may have a WDXR motif. Examples of WDXR motifs can be found in E3 substrate receptors that interact with Cullin-4A or 4B, via DDB1. Examples of WDXR motifs include those of the DCAF family of proteins (e.g., DCAF1, DCAF9 and DDB2). DDB1 interacts with CUL4 (similar to Skp1), and proteins such as DCAF1 provide the substrate recognition (similar to Skp2). DCAF1-type proteins use repeats of WD40 motifs, in which WDXR motifs are embedded, to bind to DDB1. The interactions between DDB11/WDXR proteins and E3 ligases have been studied in depth (see. e.g., Scrima et al. Cell 2008 135: 1213-1223, Yumimoto et al Journal of Biological Chemistry 2013 288: 28488-28502, Fischer et al Cell 2011 147: 1024-39, Fischer Nature 2014 512: 49-53, Schabla Journal of Molecular Cell Biology 2019 11: 725-735 and Jackson et al. Trends Biochem Sci. 2009 34: 562-570) and the sequence of this domain can be readily derived from these studies.

In alternative embodiments, the fusion protein could be a fusion between a target binding domain and an E3 ligase, such as one of the Cullins or E3 ubiquitin-protein ligase CHIP (see, e.g., Portnoff et al. J. Biol. Chem. 214 289: 7844 7855).

Finally, it may be possible to directly link the domain from Rbx1 that binds to E2 to a target binding domain. This fusion may still bind to the E3 ligase (as shown in FIG. 2) or it may bypass the E3 ligase if E2 can transfer ubiquitin onto substrates autonomously.

In any embodiment, the degradation domain, the target-binding domain and/or the linker may be selected or modified so that there are no lysines on the surface of the domain, thereby protecting the fusion protein from cis-ubiquitination and subsequent auto-degradation. In these embodiments, this domain may be designed by running a sequence through a structural prediction program, identifying lysines on the surface of a domain, and then changing the lysines to another residue (e.g., arginine, which is similar to lysine but not targeted by the ubiquitin ligase). In some embodiments, all of the lysines in one or more of the domains of the fusion protein may be modified to be arginines. In these embodiments, the fusion protein may be lysine free. In other embodiments, a subset of lysines (e.g., 1, 2, 3, 4, 5, 6 or 7 lysines) may be mutated to tune the balance of cis- versus trans-ubiquitination. These lysines may be identified based on their propensity for ubiquitination or surface accessibility. This strategy may be useful for tuning the activity of the protein degrader tool.

Linkers

In some embodiments, the fusion protein may further comprise (c), a linker, between the target-binding domain of (a) and the degradation domain of (b). A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

Suitable linkers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary linkers include glycine polymers (G)n, glycine-serine polymers (including, for example, (GS)n, (GSGGS)n (SEQ ID NO:42) and (GGGS)n (SEQ ID NO:43), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:44), GGSGG (SEQ ID NO:45), GSGSG (SEQ ID NO:46), GSGGG (SEQ ID NO:47), GGGSG (SEQ ID NO:48), GSSSG (SEQ ID NO:49), and the like.

Target-Binding Domains

In some embodiments, the target-binding domain may be antibody-based and, as such, may be a scFv or nanobody. Other antibody-based recognition domains, cAb VHH (camelid antibody variable domains) and humanized versions, IgNAR VH (shark antibody variable domains) and humanized versions, sdAb VH (single domain antibody variable domains) and “camelized” antibody variable domains are suitable for use.

In other embodiments, the target-binding domain is a non-antibody target-binding domain. In these embodiments, the target-binding domain may be an affibody; engineered Kunitz domain; a monobody (adnectin); anticalin; designed ankyrin repeat domain (DARPins); a binding site of a cysteine-rich polypeptide (e.g., cysteine-rich knottin peptides); an avimer; an afflin; and the like. See, e.g., Gebauer and Skerra (2009) Curr. Opin. Chem. Biol. 13:245. In some embodiments, a non-antibody target binding domain may comprise a SH2 domain, a SH3 domain, PDZ domains, beta-lactamase, high affinity protease inhibitors, or small disulfide binding protein scaffolds such as scorpion toxins. Methods for making binding sites derived from these molecules have been disclosed in the art, see e.g., Panni et al., J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, a binding domain derived from tetranectin in its monomeric or trimeric form, and other such domains known to those of ordinary skill in the art, as well as derivatives and/or variants thereof. Exemplary non-antibody-based scaffolds, and methods of making the same, can also be found in Stemmer et al., “Protein scaffolds and uses thereof”, U.S. Patent Publication No. 20060234299 (Oct. 19, 2006) and Hey, et al., Artificial, Non-Antibody Binding Proteins for Pharmaceutical and Industrial Applications, TRENDS in Biotechnology, vol. 23, No. 10, Table 2 and pp. 514-522 (October 2005). In some embodiments, the target binding domain may be a “synZIP”, which are heterospecific synthetic coiled-coil peptides that bind to one another in a pairwise manner (see, e.g., Keating et al. ACS Synth. Biol. 2012 1: 118-29).

In some embodiments, the target binding domain may bind to a post-translationally modified motif. In these embodiments, the target-binding domain comprises an SH2 domain or PTB domain (which bind to motifs that have a phosphotyrosine), a FHA, or WD40-repeat domain (which can bind to motifs that contain phosphoserine or phosphothreonine), a bromo domain (which bind to motifs that have an acylated lysine) or a chromo domain (which bind to motifs that have an methylated lysine). These latter embodiments allow one to degrade signaling proteins, but only when they are in the process of signaling.

Transmembrane Domains

In some embodiments, the fusion protein may have a transmembrane domain. In these embodiments, the fusion protein may target a transmembrane protein such as a CAR, SynNotch, a receptor, or any other protein that is located on the plasma membrane of a mammalian cell (see, e.g., Sharpe et al, Cell. 2010 142: 158-169). In these embodiments, the fusion protein may comprise: (a) a transmembrane domain, (b) a target-binding domain that binds to an intracellular site in a transmembrane protein (such as a CAR, SynNotch or a receptor); and (c) a degradation domain that is heterologous to the target-binding domain, wherein the degradation domain is a degron or E3 ligase-recruiting domain, as discussed above. When the protein is expressed in a mammalian cell, the target-binding domain and degradation domain are intracellular, but tethered to the plasma membrane via the transmembrane domain. In the cell, binding of the fusion protein to the transmembrane protein via the target-binding domain induces degradation of the transmembrane protein, as discussed above. As would be apparent, the nucleic acid encoding such a fusion protein may additionally comprise a signal peptide. Suitable transmembrane domains include those of CD8, CD4, CD3 zeta, CD28, CD134, CD7, although there are thousands of others that one could use. The transmembrane domain can be C-terminal or N-terminal, or anywhere in the fusion protein depending on the other components of the protein used.

In these embodiments, the fusion protein can be used to controllably target a membrane protein such as a CAR or SynNotch in a CAR T cell. In some embodiments, the fusion protein could be part of a circuit that controls the expression of a CAR. For example, expression of the fusion protein could be induced by binding of a first antigen to a binding triggered transcriptional switch such as a SynNotch. After the switch is activated, the fusion protein is expressed, and the fusion protein degrades a CAR in the cell. This way, the CAR expression can be “switched off” by binding of the SynNotch to the first antigen.

Target Proteins

The target protein can be endogenous (i.e., native) to the cell or exogenous to the cell (i.e., expressed using recombinant means). Examples of target proteins include, but are not limited to, transcriptional activators, transcriptional repressors, transcriptional co-activators, transcriptional co-repressors, DNA binding polypeptides, RNA binding polypeptides, translational regulatory polypeptides, hormones, cytokines, toxins, antibodies, chromatin modulators, suicide proteins, organelle specific polypeptides (e.g., a nuclear pore regulator, a mitochondrial regulator, an endoplasmic reticulum regulator, and the like), pro-apoptosis polypeptides, anti-apoptosis polypeptides, other polypeptides that promote cell death through other mechanisms, pro-proliferation polypeptides, anti-proliferative polypeptides, immune co-stimulatory polypeptides, site-specific nucleases, recombinases, inhibitory immunoreceptors, an activating immunoreceptor, Cas9 and variants of RNA targeted nucleases, and DNA recognition polypeptides, dominant negative variants of a polypeptide, a signaling polypeptide, a receptor tyrosine kinase, a non-receptor tyrosine kinase, a polypeptide that promotes differentiation, enzymes, structural proteins, and the like.

For example, in some embodiments, the target protein may be a therapeutic protein that, when expressed on the surface of an immune cell, activates the immune cell or inhibits activation of the immune cell when it binds to a third antigen on the diseased cell. In these embodiments, the therapeutic protein may be a chimeric antigen receptor (CAR) or a T cell receptor (TCR). In these embodiments, the fusion protein may comprise: (a) a target-binding domain that binds to a CAR or TCR and (b) a degradation domain that is heterologous to the target-binding domain, wherein the degradation domain is a degron or E3 ligase-recruiting domain, wherein, in the therapeutic cell, binding of the fusion protein to a target protein via the target-binding domain induces degradation of the target protein. Alternatively, the therapeutic protein may be an inhibitory immune cell receptor (iICR) such as an inhibitory chimeric antigen receptor (iCAR), wherein binding of the iICR to the third antigen inhibits activation of the immune cell on which the iICR is expressed. Such iICR proteins are described in e.g., WO2017087723, Fedorov et al. (Sci. Transl. Med. 2013 5: 215ra17) and other references cited above, which are incorporated by reference for that description and examples of the same. In some embodiments, an inhibitory immunoreceptor may comprise an intracellular immunoreceptor tyrosine-based inhibition motif (ITIM), an immunoreceptor tyrosine-based switch motif (ITSM), an NpxY motif, or a YXXΦ D motif. Exemplary intracellular domains for such molecules may be found in PD1, CTLA4, BTLA, CD160, KRLG-1, 2B4, Lag-3, Tim-3 and other immune checkpoints, for example. See, e.g., Odorizzi and Wherry (2012) J. Immunol. 188:2957; and Baitsch et al. (2012) PLoSOne 7: e30852.

In some embodiments, therapeutic protein may be an antigen-specific therapeutic that is secreted from the cell. For example, the antigen-specific therapeutic may be an antibody that binds to an immune checkpoint inhibitor e.g., an antibody that binds to PD1, PD-L1, PD-L2, CTLA4, TIM3, LAG3 or another immune checkpoint.

Alternatively, the secreted antigen-specific therapeutic may be a bioactive peptide such as a cytokine (e.g., I1-1ra, IL-4, IL-6, IL-10, IL-11, IL-13, or TGF-β, among many others). In some embodiments, the secreted protein may be an enzyme, e.g., a superoxide dismutase for removing reactive oxygen species, or a protease for unmasking a protease activatable antibody (e.g., a pro-body) in the vicinity of a cancer cell.

Alternatively, the therapeutic protein may be a protein that, when expressed, is internal to the cell, such as wild type or mutant SLP76, ZAP70, or Cas9 protein.

If the target protein is endogenous, then the target-binding domain of the fusion protein may contain a domain of a natural binding partner of the target protein, or another specific binding domain such as a nanobody or scFv.

If the target protein is exogenous, then in some cases the target protein can be engineered to contain a binding site for the target-binding domain of the fusion protein. In these embodiments, the target protein can be designed to contain an epitope tag (e.g., a hemagglutinin, FLAG, c-myc, ALFA, or V5 tag), and the like to which the target-binding domain binds. Alternatively, the target protein can be designed to contain a synthetic leucine zipper domain that heterodimerizes with a complementary synthetic leucine zipper domain in the fusion protein (see, e.g., Keating et al. ACS Synth. Biol. 2012 1: 118-29). For example, one could knock-in a binding site (i.e., a binding site for the target-binding domain of the fusion protein) into an endogenous locus such that, when the protein of interest contains the binding site when it is expressed. This strategy may be employed if an the target protein does not have an endogenous binding partner or scFv/nanobodies that bind to the target protein are not available. For example, if the protein of interest is the PD-1, one could knock-in a SynZIP into the endogenous PD-1 locus so that the expression of the endogous PD-1 protein can be regulated using the present fusion protein.

In some cases, binding of the fusion protein to the target protein may be conditional. In these embodiments, target binding domain of the fusion protein and the target protein may be engineered to only bind to one another in the presence of dimerization agent. Examples of pairs of protein domains that conditionally dimerize with one another include: FKBP and FKBP (which dimerize in the presence of rapamycin), FKBP and CnA (which dimerize in the presence of rapamycin), FKBP and cyclophilin (which dimerize in the presence of rapamycin), FKBP and FRG (which dimerize in the presence of rapamycin), GyrB and GyrB (which dimerize in the presence of coumermycin), DHFR and DHFR (which dimerize in the presence of methotrexate), DmrB and DmrB (which dimerize in the presence of AP20187), PYL and ABI (which dimerize in the presence of abscisic acid), Cry2 and CIB1 (which dimerize in the presence of blue light); GAI and GID1 (which dimerize in the presence of gibberellin) and a ligand-binding domain of a nuclear hormone receptor, and a co-regulator of the nuclear hormone receptor (which dimerize in the presence of a nuclear hormone, agonists thereof and antagonists thereof, e.g., tamoxifen). In embodiments in which rapamycin can serve a dimerizer, a rapamycin derivative or analog can also be used.

In any embodiment, the fusion protein may contain a localization signal (e.g., a nuclear localization sequence) in order to facilitate translocation of the fusion into a cell compartment (e.g., the nucleus).

In any embodiment, expression of the fusion protein may be inducible, tissue-specific, or constitutive. This may be done by operably linking the coding sequence for the fusion protein to an appropriate promoter.

The therapeutic cell may be genetically modified to contain a nucleic acid comprising an expression cassette comprising a promoter and a coding sequence for the fusion protein as described above. The therapeutic cell may be an immune cell or stem cell, for example, and the nucleic acid may be introduced into the cell by various means, including e.g., through the use of a viral vector.

As noted above, in some embodiments, the therapeutic cell may also express a therapeutic protein, where the therapeutic protein may be on the surface of the cell, secreted by the cell, or on the inside of the cell (e.g., in the cytoplasm or nucleus of the cell).

In some instances, a therapeutic cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or a progenitor thereof, obtained from an individual. As another example, the cell is a cytotoxic cell, or a progenitor thereof, obtained from an individual. As another example, the cell is a stem cell or progenitor cell obtained from an individual.

In some cases, the cell is an immune cell, e.g., a T cell, a B cell, a macrophage, a dendritic cell, a natural killer cell, a monocyte, etc. In some cases, the cell is a T cell. In some cases, the cell is a cytotoxic T cell (e.g., a CD8+T cell). In some cases, the cell is a helper T cell (e.g., a CD4+T cell). In some cases, the cell is a regulatory T cell (“Treg”). In some cases, the cell is a B cell. In some cases, the cell is a macrophage. In some cases, the cell is a dendritic cell. In some cases, the cell is a peripheral blood mononuclear cell. In some cases, the cell is a monocyte. In some cases, the cell is a natural killer (NK) cell. In some cases, the cell is a CD4+, FOXP3+Treg cell. In some cases, the cell is a CD4+, FOXP3-Treg cell. The immune cell can be immunostimulatory or immunoinhibitory.

In some embodiments, the therapeutic cell may be a CAR T cell. In these embodiments, the cell may be a T cell that expresses a CAR, where the CAR comprises an extracellular domain, a transmembrane region and an intracellular signaling domain; where the extracellular domain comprises a ligand or a receptor and the intracellular signaling domain comprises an ITAM domain, e.g., the signaling domain from the zeta chain of the human CD3 complex (CD3zeta), and, optionally, one or more costimulatory signaling domains, such as those from CD28, 4-1BB and OX-40. The extracellular domain contains a recognition element (e.g., an antibody or other target-binding scaffold) that enables the CAR to bind a target. In some cases, a CAR comprises the antigen binding domains of an antibody (e.g., an scFv) linked to T-cell signaling domains. In some cases, when expressed on the surface of a T cell, the CAR can direct T cell activity to those cells expressing a receptor or ligand for which this recognition element is specific. As an example, a CAR that contains an extracellular domain that contains a recognition element specific for a tumor antigen can direct T cell activity to tumor cells that bear the tumor antigen. The intracellular region enables the cell (e.g., a T cell) to receive costimulatory signals. The costimulatory signaling domains can be selected from CD28, 4-1BB, OX-40 or any combination of these. Exemplary CARs comprise a human CD4 transmembrane region, a human IgG4 Fc and a receptor or ligand that is tumor-specific, such as an IL13 or IL3 molecule. In these embodiments, activation of a CAR activates the immune cell.

Suitable therapeutic cells also include stem cells, progenitor cells, as well as partially and fully differentiated cells. Suitable cells include neurons; liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); and a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

Cells of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, an immune cell, a stem cell, etc., is obtained from an individual; and the cell obtained from the individual is modified to express components of a circuit of the present disclosure. The modified cell can thus be modified with control feedback to one or more signaling pathways of choice, as defined by the one or more molecular feedback circuits present on the introduced nucleic acids. In some cases, the modified cell is modulated ex vivo. In other cases, the cell is introduced into and/or already present in an individual (e.g., the individual from whom the cell was obtained); and the cell is modulated in vivo, e.g., by administering a nucleic acid or vector to the individual in vivo.

In some instances, the cell is obtained from an individual. For example, in some cases, the cell is a primary cell. As another example, the cell is a stem cell or progenitor cell obtained from an individual.

As one non-limiting example, in some cases, the cell is an immune cell obtained from an individual. As an example, the cell can be a T lymphocyte obtained from an individual. As another example, the cell is a cytotoxic cell (e.g., a cytotoxic T cell) obtained from an individual. As another example, the cell can be a helper T cell obtained from an individual. As another example, the cell can be a regulatory T cell obtained from an individual. As another example, the cell can be an NK cell obtained from an individual. As another example, the cell can be a macrophage obtained from an individual. As another example, the cell can be a dendritic cell obtained from an individual. As another example, the cell can be a B cell obtained from an individual. As another example, the cell can be a peripheral blood mononuclear cell obtained from an individual.

In some cases, the host cell is not an immune cell. In these embodiments, the host cell may be a somatic cell, e.g. a fibroblast, a hematopoietic cell, a neuron, a pancreatic cell, a muscle cell, a bone cell, a hepatocyte, a pancreatic cell, an epithelial cell, an endothelial cell, a cardiomyocyte, a T cell, a B cell, an osteocyte, or a stem cell, and the like.

Given that the genetic code is known, sequence that encodes the fusion protein can be readily determined. In some embodiments, the coding sequence may be codon optimized for expression in mammalian (e.g., human or mouse) cells, strategies for which are well known (see, e.g., Mauro et al., Trends Mol. Med. 2014 20: 604-613 and Bell et al Human Gene Therapy Methods 27: 6). As would be understood, the coding sequence may be operably linked to a promoter, which may be inducible, tissue-specific, or constitutive. In some embodiments, the promoter may be activated by an engineered transcription factor that is heterologous to the cell, e.g., a Gal4-, LexA-, Tet-, Lac-, dCas9-, zinc-finger- and TALE-based transcription factors.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natd. Acad. Sci. USA 90: 7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

In some cases, the promoter is a cardiomyocyte-specific promoter. In some cases, the promoter is a smooth muscle cell-specific promoter. In some cases, the promoter is a neuron-specific promoter. In some cases, the promoter is an adipocyte-specific promoter. Other cell type-specific promoters are known in the art and are suitable for use herein.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

The cell may be used in a method of treatment that comprises administering the cell to a patient in need thereof.

In some embodiments, the patient may have cancer, e.g., breast cancer, B cell lymphoma, pancreatic cancer, Hodgkin lymphoma cell, ovarian cancer cell, prostate cancer, mesothelioma, lung cancer (e.g., a small cell lung cancer), non-Hodgkin B-cell lymphoma (B-NHL) cell, ovarian cancer, a prostate cancer, melanoma cell, a chronic lymphocytic leukemia cell, acute lymphocytic leukemia cell, a neuroblastoma, a glioma, a glioblastoma, a medulloblastoma, a colorectal cancer, etc. In these embodiments, the therapeutic cell may be a CAR T cell that comprises a CAR that recognizes an antigen expressed by the cancer cells.

In some embodiments, the patient may have an inflammatory condition or autoimmune disease. In these embodiments, the cell may be T-helper cell or a Tregs for use in an immunomodulatory method. Immunomodulatory methods include, e.g., enhancing an immune response in a mammalian subject toward a pathogen; enhancing an immune response in a subject who is immunocompromised; reducing an inflammatory response; reducing an immune response in a mammalian subject to an autoantigen, e.g., to treat an autoimmune disease; and reducing an immune response in a mammalian subject to a transplanted organ or tissue, to reduce organ or tissue rejection.

In some embodiments, the patient is in need of a stem cell transplantation.

Plasma Membrane Embodiments
1. A fusion protein comprising: (a) a transmembrane domain; (b) a target-binding domain that binds to an intracellular site in a transmembrane protein; and (c) a degradation domain that is heterologous to the target-binding domain, wherein the degradation domain is a degron or E3 ligase-recruiting domain; wherein, in a cell, the target-binding domain and degradation domain are intracellular and binding of the fusion protein to the transmembrane protein via the target-binding domain induces degradation of the transmembrane protein.
2. The fusion protein of embodiment 1, wherein the fusion protein further comprises (c), a linker, between the target-binding domain of (a) and the degradation domain of (b).
3. The fusion protein of any prior embodiment, wherein the degradation domain is a degron.
4. The fusion protein of any of embodiments 1-2, wherein the degradation domain is an E3 ligase-recruiting domain.
5. The fusion protein any prior embodiment, wherein there are no lysines on the surface of the E3 ligase-recruiting domain and/or the target binding domain.
6. The fusion protein of any prior embodiment, wherein the target-binding domain is a scFv or nanobody.
7. The fusion protein of any prior embodiment, wherein the target-binding domain is a non-antibody target-binding domain.
8. The fusion protein of any prior embodiment, wherein the target-binding domain binds to a motif having a post-translational modification.
10. The fusion protein of any prior embodiment, wherein the transmembrane protein is a CAR.
11. The fusion protein of any of embodiments 1-9, wherein the transmembrane protein is a synNotch.
12. The fusion protein of any prior embodiment, wherein the target-binding domain is a first synthetic leucine zipper the transmembrane protein comprises a second synthetic leucine zipper, wherein the first and second leucine zippers interact.
13. A nucleic acid encoding a fusion protein of any of embodiment 1-12.
14. A construct comprising a promoter and the coding sequence of embodiment 13, wherein the promoter and coding sequence are operably linked.
15. The construct of embodiment 14, wherein the promoter is inducible, constitutive, cell type-specific or tissue specific.
16. A cell comprising a construct of any of embodiments 14 and 15, wherein the fusion protein is expressed in the cell and, in the cell, the target-binding and degradation domains are intracellular and binding of the fusion protein to a transmembrane protein in the cell via the target-binding domain induces degradation of the transmembrane protein.
17. The cell of embodiment 16, wherein the target protein is endogenous to the cell.
18. The cell of embodiment 16, wherein the target protein is exogenous to the cell.
19. The cell of embodiment 18, wherein binding of the fusion protein to the target protein is chemically inducible.
20. The therapeutic cell of any of embodiments 16-19, wherein the cell is an immune cell.
21. The therapeutic cell of embodiment 20, wherein the cell is primary T cell.
22. The therapeutic cell of embodiment 20, wherein the is a CAR T cell and the target protein is a CAR.
25. A method comprising: administering a cell of any of embodiments 1-22 to a patient in need thereof.
26. The method of embodiment 25, wherein the patient has cancer.

### EXAMPLES

The following examples are put forth so as to provide those of ordinary skill in the art with a complete disclosure and description of how to make and use the present invention, and are not intended to limit the scope of what the inventors regard as their invention.

This disclosure provides a new protein degradation technology based on a protein chimera contains a protein targeting domain, an optional linker, and a protein degradation domain, e.g., a degron. This protein chimera is able to recruit the endogenous E3 ligase machinery of the cell to novel targets, triggering the ubiquitination and degradation of natural and unnatural targets. This tool is referred to as a “synthetic targetter of ubiquitination and degradation”, or “STUD” for short. A particularly potent C-terminal minimal degron motif of the sequence RRRG (Arg-Arg-Arg-Gly; also referred to as the “Bonger” motif; SEQ ID NO:32) was used as a basis for developing this technology. In theory, this system should be amenable to a variety of degron motifs or E3 scaffold domains.

### Example 1

Cis-Ubiquitination can be Prevented by Substituting the Lysines in a STUD

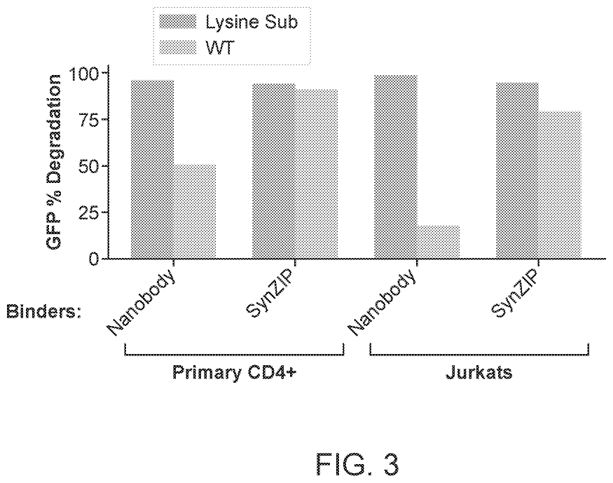

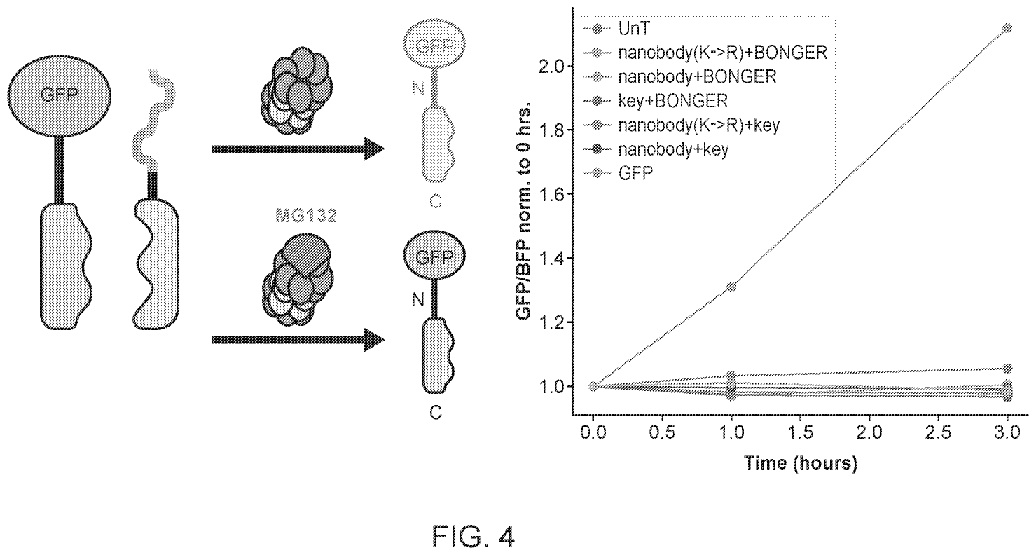

This protein degradation tool has the potential to ubiquitinate target lysines on both the target of interest (trans-ubiquitination), as well as on the tool itself (cis-ubiquitination). cis-ubiquitination may limit the effectiveness of the STUD by degrading the STUD before it has the chance to interact with its target. To solve this problem, the lysines on the protein targeting domain of the STUD were mutated to arginines (K->R), thus preventing cis-ubiquitination². An assay was developed to test the functionality of a STUD by measuring degradation of a cystosolic GFP. The GFP was targeted for degradation using either a GFP nanobody or a SynZIP17 that was fused to the GFP. The target GFP was transduced into either Jurkat cells or primary human T cells using lentivirus and the STUD was introduced via a second lentivirus. It was observed that the lysine substitution significantly improved the activity of the GFP nanobody STUD, whereas the mutation only moderately improved the activity of the SynZIP STUD. These results are shown in FIG. 3. This trend was consistent between primary human CD4+T cells and Jurkats. Given these results, it should be possible to use the number of lysines on the STUD as a strategy for tuning the activity of the STUD, where more mutated lysines increases the activity of the STUD.

### Example 2

STUD-Induced Degradation is Mediated Via the Proteasome

The mechanism of how the STUD reduces GFP was explored. Primary human CD4+T cells expressing the GFP nanobody STUD were fed with the MG132 proteasome inhibitor and the change in fluorescence was measured over time. These results are shown in FIG. 4. Cells expressing a functional STUD should display an increase in fluorescence over time as the proteasome inhibitor took effect. After three hours of exposure to the drug, it was observed that only the cells expressing the functional nanobody STUD (nanobody(K->R)+Bonger) displayed an increase in GFP fluorescence. This indicates that the observed reduction in GFP is mediated by degradation via the proteasome rather than a mechanism associated with the protein-protein interaction alone.

### Example 3

STUD Activity can be Optimized Using a Linker

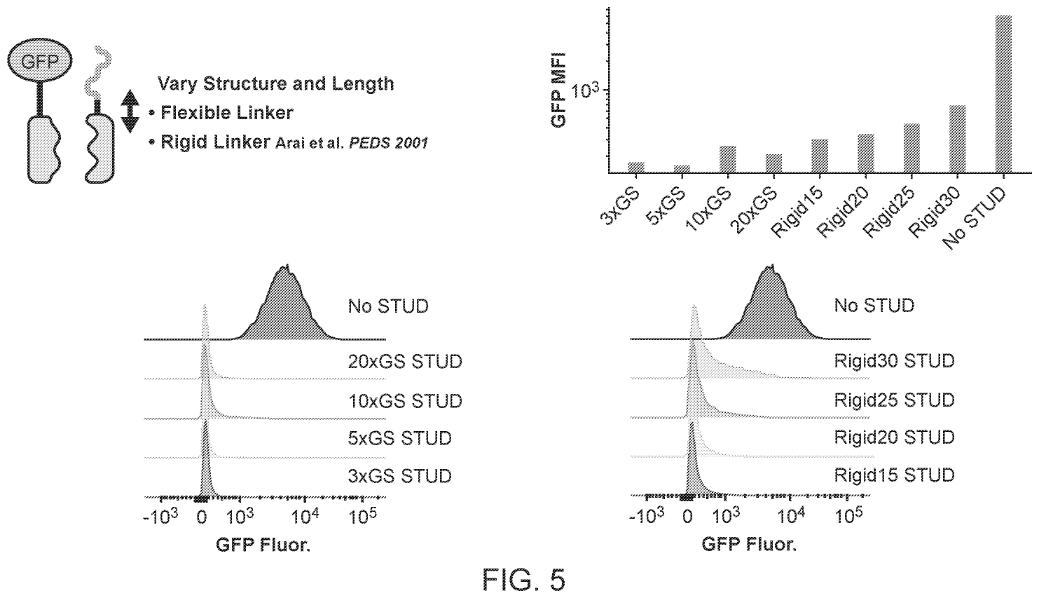

The STUD was optimized by screening multiple lengths of two different classes of linkers. In these constructs, the linker was added between a SynZIP protein binding domain and the Bonger degron. It was hypothesized that a flexible Gly-Ser linker may facilitate target degradation by increasing the accessibility of the E3 ligase to reach target lysine residues on the surface of the target protein, whereas a rigid helical linker may increase the distance between the E3 ligase and target lysines and reduce degradation. These experiments used the SynZIP STUD that targets cytosolic GFP-SZ17 as described above. Four lengths of linker for both the flexible and rigid linker. The flexible linker generally performed better than the rigid linker, with little variation in degradation efficiency observed within the different flexible linker lengths (FIG. 5). However among the flexible linkers the 5xGS performed the best. This STUD (with the SynZIP(K->R), optimized linker and C-terminal RRRG (SEQ ID NO:32), or SynZIP18(K->R)-5xGS-RRRG; SEQ ID NO:32) is referred to as the “soluble stud” and used in the following experiments.

### Example 4

Transcription Factors can be Targeted

Lysine substitution and linker length/type optimization served as a framework for optimizing future STUD iterations that use other protein targeting domains and/or degradation domains, e.g., degrons. Depending on the application, different synthetic protein targeting domains may be more suitable, and it is also possible to utilize endogenous protein targeting domains that bind to or interact with an endogenous protein without the need for modification of the endogenous protein. Furthermore, different degrons may be utilized to vary the conditions under which the STUD is active, or confine the activity of the STUD to different compartments of the cell where the degron is active.

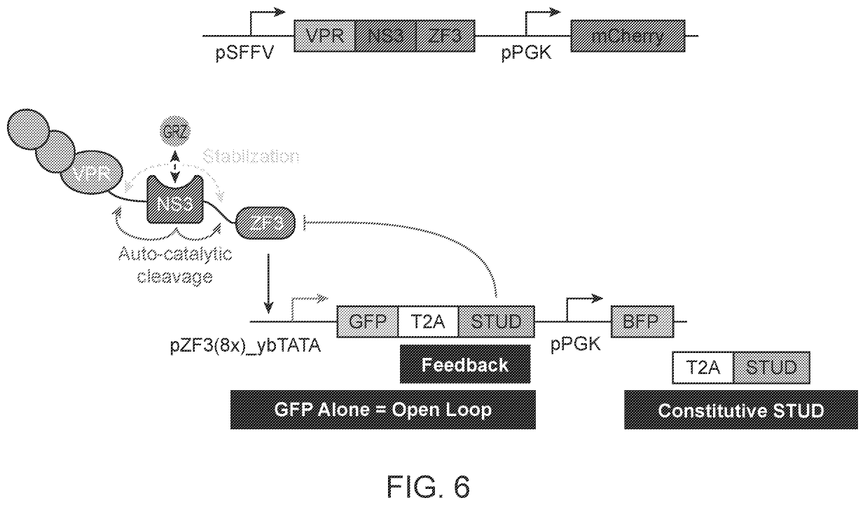

A transcription factor was targeted for degradation using the soluble STUD described above. Modulating a transcription factor allows one to affecting the output of a functional protein. Thee experiments were done using a previously developed grazoprevir (GRZ) drug-inducible zinc-finger transcription factor system (VPR-NS3-ZF3). To induce degradation of this transcription factor SynZIP17 to the C-terminus of this protein. Degradation of the TF was measured by observing changes in GFP reporter output driven by the pZF3(8×)ybTATA promoter. Two different methods were used for STUD expression: constitutive STUD expression, or inducible STUD expression, which should drive negative feedback in the system (FIG. 6).

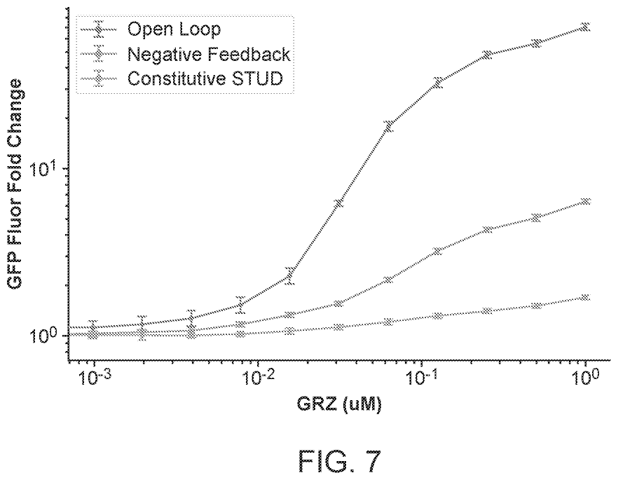

The dose responses of the three circuit variants were compared to assess the functionality of the STUD. It was found that constitutive expression of the STUD abolished nearly all output from the pZF3, whereas feedback expression of the STUD generated an intermediate dose response (FIG. 7). This demonstrates that the soluble STUD can not only degrade functional proteins in the cell, but also be used as a powerful tool for building genetic circuits.

### Example 5

Transmembrane Proteins can be Targeted

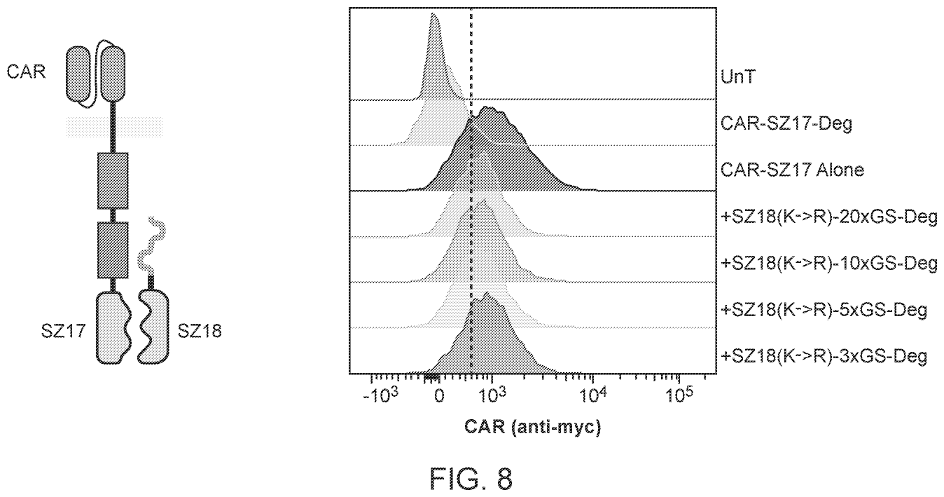

Next, the soluble STUD was used to target a membrane protein for degradation. The ability of the STUD to degrade a CAR in Jurkat cells was tested by generating a CAR construct with SynZIP17 fused to its C-terminus. However, while these STUDs worked to some extent, none of them were able to completely knockdown CAR expression (see FIG. 8).

It was found that the Bonger degron, when directly fused to the CAR, was able to reduce CAR expression by over 90%. This result suggested that the soluble STUD was not working due to insufficient interaction with the CAR, rather than a defect with the ability of the degron to target membrane proteins for degradation.

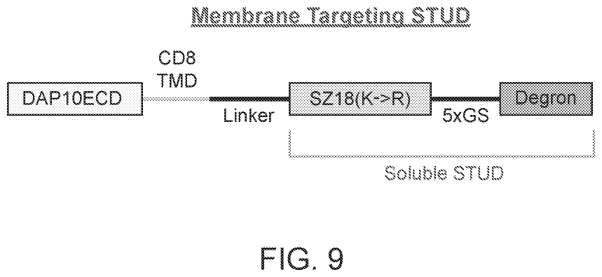

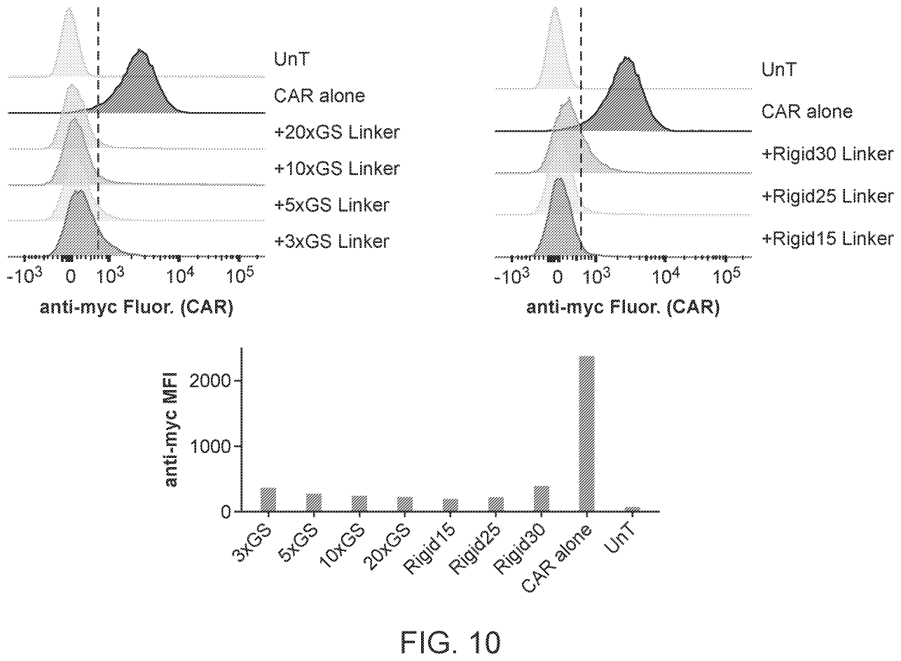

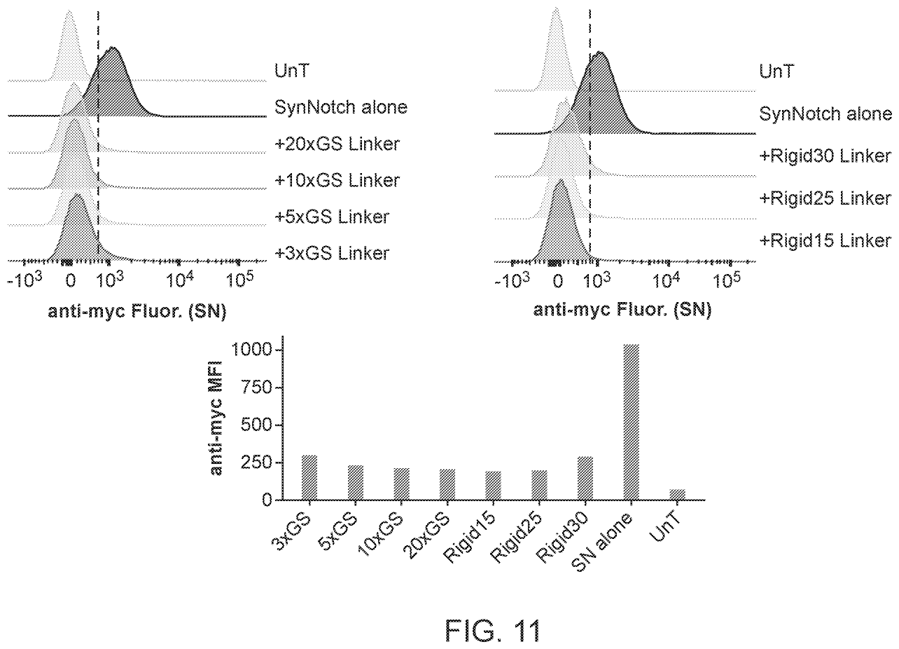

To increase the likelihood of interaction between the STUD and the CAR, a new STUD construct that was itself localized to the membrane using the DAP10 signal sequence was generated (FIG. 9). A library of linkers between the CD8 transmembrane domain and the soluble STUD was also tested. The ability of these new membrane targeting STUDs were tested for their ability to degrade both a CAR and a SynNotch in primary human CD4+T cells. It was found that the best results were provided using a rigid linker between the CD8 TMD and STUD. All linkers were effective, but use of the Rigid15 linker resulted in over 95% knock-down of CAR expression as measured by surface staining for CAR expression (FIG. 10). This result was replicated for a membrane targeting STUD targeting a SynNotch for degradation (FIG. 11).

### Example 6

Synthetic Tarmeter of Ubiquitination and Degradation (‘STUD’) Potently Degrades Fluorescent Protein Targets in all Tested Mammalian Cell Lines

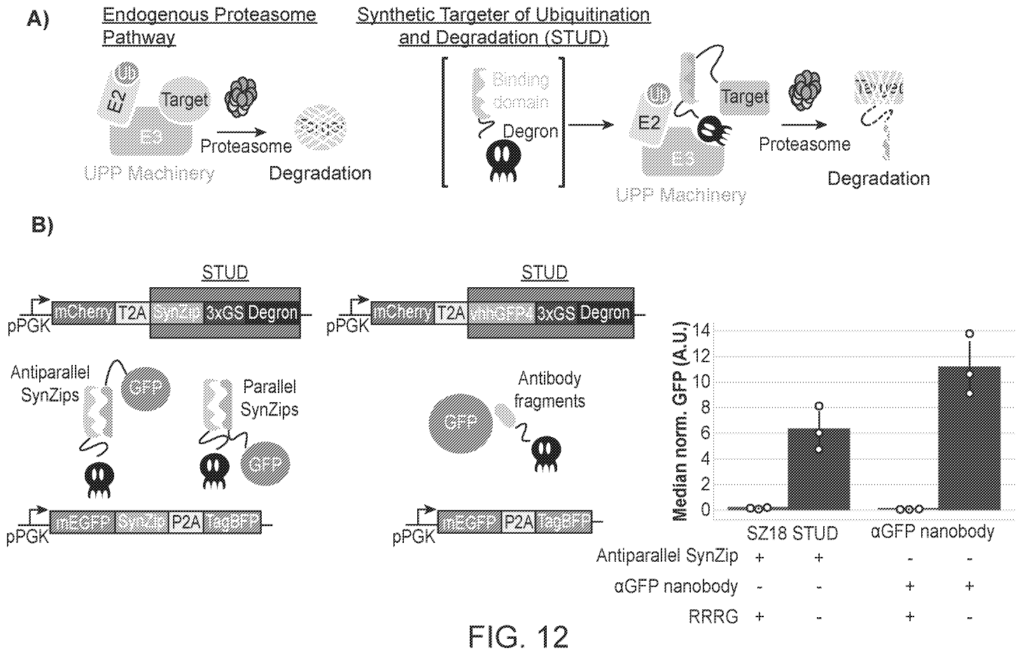

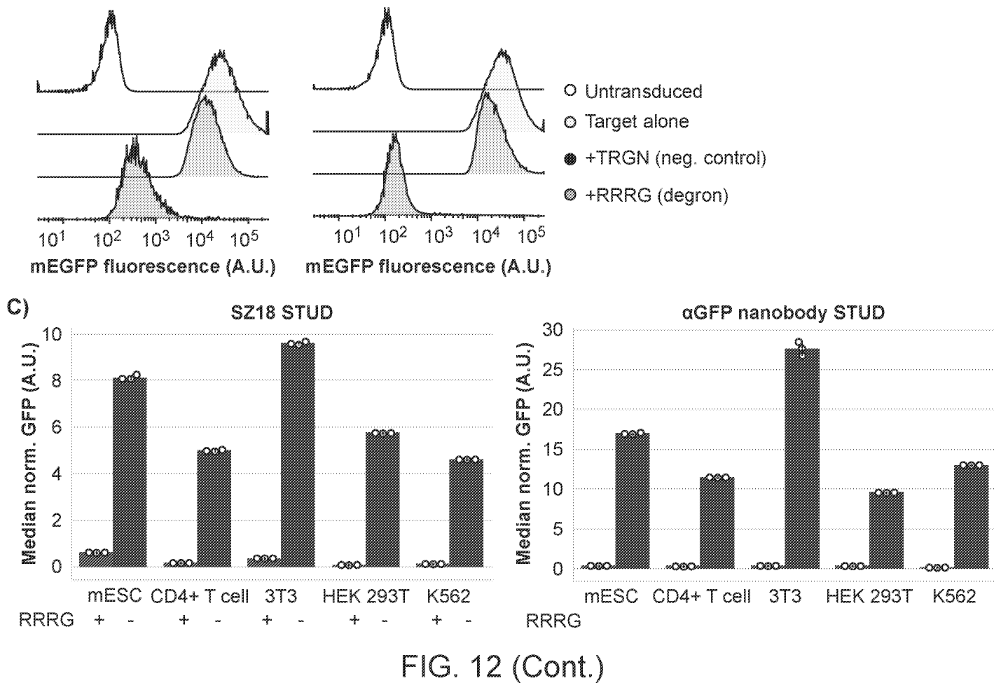

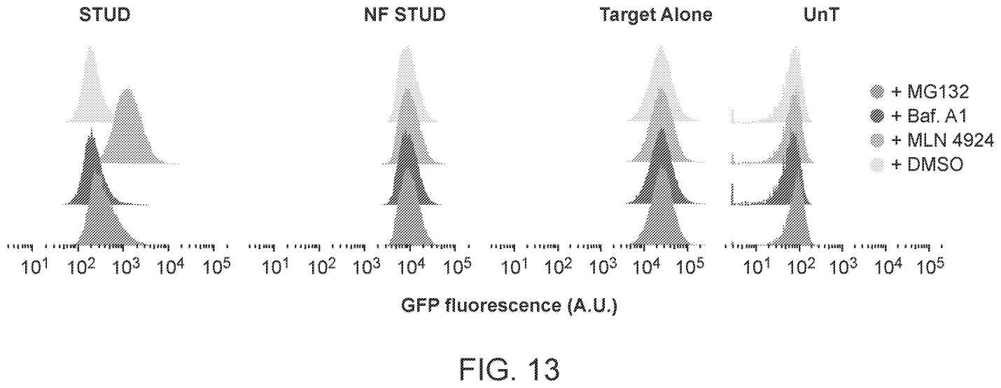

A new synthetic degradation molecule, referred to as a Synthetic Targeter of Ubiquitination and Degradation (‘STUD’), was designed. A STUD is composed of a binding domain that specifically identifies and dimerizes with target molecules and a degradation domain to recruit the UPP machinery to induce ubiquitination and subsequent degradation by the proteasome. Through these domains, STUDs act as a molecular bridge between the ubiquitin conjugation machinery of the UPP and a specific protein target of interest (FIG. 12A). Here STUD modularity is demonstrated with a minimal toolbox that consists of two orthogonal binding domains and two degradation domains. To emphasize the potential compactness of this system, previously described heterodimeric synthetic leucine zipper proteins were used (see Thompson et al supra 2012). It has also been demonstrated that STUDs can target proteins with minimal changes to their endogenous sequence using a nanobody that binds green fluorescent protein (GFP) (Saerens J. Mol. Bio. 2005 352: 597-607). The degradation domain of choice for the STUDs shown in this work is a minimally sufficient sequence (‘+RRRG’; SEQ ID NO:32) from the FKBP degron that was previously described (Bonger 2011, supra). From this same work, a similar sequence with minimal observed degradation as a non-functional control was identified, which is referred to as ‘+TRGN’ (SEQ ID NO:50) or ‘-RRRG’ (SEQ ID NO:32) interchangeably.

Either the SynZip STUD and the antiGFP nanobody STUD in Jurkat T cells alongside a plasmid encoding either GFP fused to a complementary SynZip or GFP alone, respectively (FIG. 1B) were lentivirally transduced. 72 hours following removal of lentivirus, we assay for GFP fluorescence by flow cytometry. To quantify STUD degradation efficacy, we isolate cells by gating out cells with fluorescence values for both cotransduction markers less than those of untransduced (‘UnT’) Jurkat T cells. Normalized GFP fluorescence was calculated by normalizing individual cell GFP fluorescence by tagBFP fluorescence to account for any differences due to variations in plasmid expression or integration copy number. Looking at the median of the distributions of this normalized GFP for each condition, an approximately 42-fold change with the SynZip STUD and an approximately 167-fold change with the nanobody STUD was observed. Representative histograms of unnormalized GFP fluorescence are also shown for reference.

The potential wider application of STUDs as a tool for mammalian synthetic biology was demonstrated by replicating potent GFP degradation in other cell lines. For adherent cell lines (human embryonic (HEK) 293T, 3T3, and mouse embryonic stem cells (mESCs)), cells were seeded 24 hours before lentiviral transduction. While for suspension cell lines (K562 myelogenous leukemia cells and primary human CD4+T cells) are plated the same day as a lentiviral addition. Experimental design following lentiviral addition is the same as for Jurkat T cells. Using the same analysis method as described above, it was observed that the degradation capability of STUDs is similarly efficacious across all tested cell lines.

### Example 7

Loss in Target Signal can be Rescued by Inhibition of UPP

Inhibitors of the proteasomal and lysosomal degradation pathways were used to demonstrate that loss in GFP fluorescence can be attributed to degradation. Using the 2 plasmid system as described above, we lentivirally transduce Jurkat T cells. 72 hours after removal of lentivirus, we treat these Jurkat T cells and an untransduced control cell line with either 5 μM of the proteasomal inhibitor MG-132, 1 μM of the cullin ring ligase inhibitor MLN4924, 100 nM of the lysosomal inhibitor Bafilomycin Al, or DMSO vehicle control and incubate at 37 C for 5 hours. Using flow cytometry to measure GFP fluorescence following treatment, we observe that GFP fluorescence can indeed be rescued with MG-132 and MLN4924 when cells express both a functional STUD and a GFP target relative to DMSO vehicle control. No changes to GFP fluorescence were seen with bafilomycin treatment. Similar GFP fluorescence values were observed in cells expressing either GFP target and a non-functional STUD or GFP alone across all conditions. Together, these data show that loss of GFP fluorescence in the presence of a STUD is due to degradation and that this degradation is mediated by the proteasome.

### Example 8

Tethering of STUD to Plasma Membrane Allows for Functional Knockdown for Second-Generation Chimeric Antigen Receptors

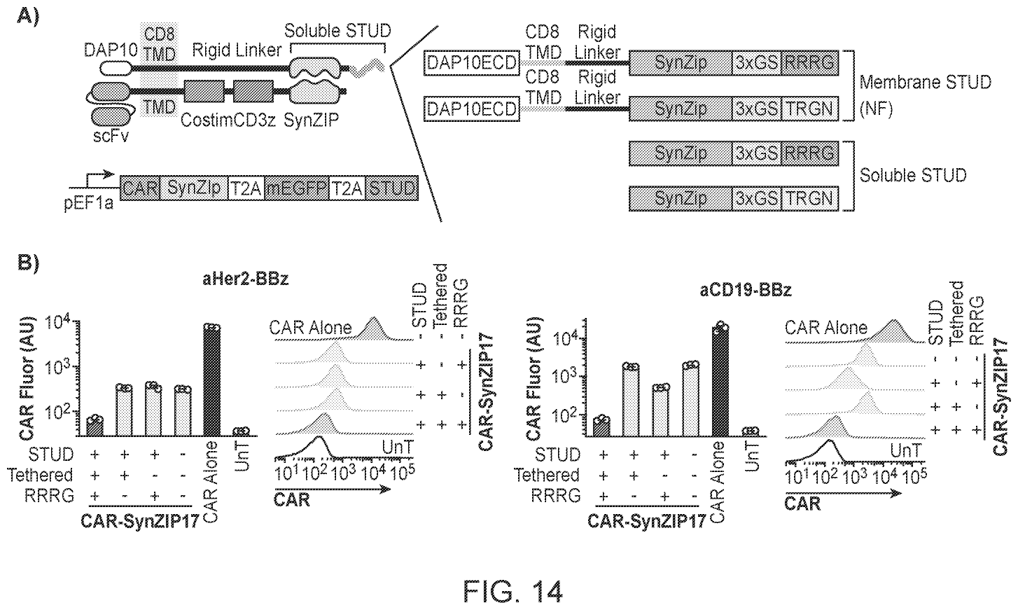

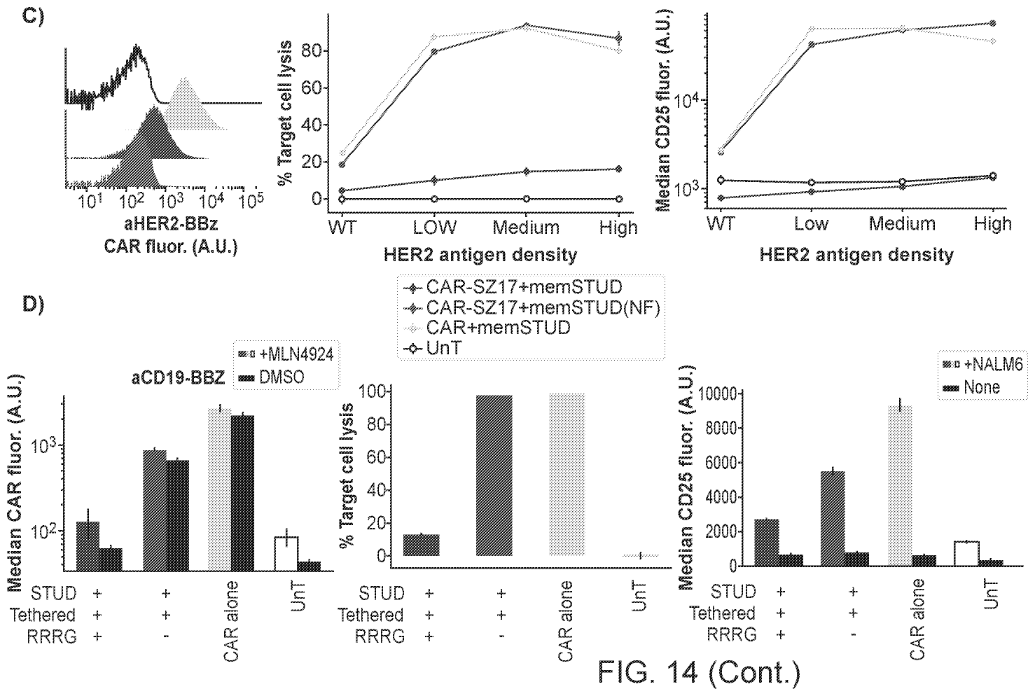

In initial tests, it was found that a STUD alone only degraded membrane proteins, namely a chimeric antigen receptor (CAR), inefficiently. Increasing the local concentration of the STUD at the membrane by fusion to a membrane localization domain was tested. The STUD was fused to a previously published membrane localization domain consisting of a truncated extracellular domain from the DAP10 protein and a transmembrane domain from the CD8 alpha protein (Wu 2015). The plasmids were lentivirally transduced into cells encoding this new membrane-tethered STUD (‘memSTUD’), a variant of the memSTUD with the non-functional sequence used in previous figures, or the original version of the STUD described in previous figures (‘soluble STUD’) along with a second-generation CAR and a GFP transduction marker (FIG. 14A). The ability of the memSTUDs to degrade two 4-1BB variant second-generation (‘BBz’) CARs that target CD19 or HER2 in Jurkat T cells was tested. 72 hours after removal of lentivirus, an antibody stain specific for an extracellular myc tag fused to the CAR was used. The surface CAR expression by fluorescence of this antibody stain was measured by flow cytometry. It was observed that memSTUDs are able to potently degrade both types of 4-1BB CARs (FIG. 14B).

Next, CD8+primary human T cells were lentivirally transduced with the same constructs as described above, isolated populations of interest by FACS, and co-cultured these cell populations with target cells expressing either a CAR antigen or no antigen for 72 hours. For HER2BBz CARs, we coculture engineered CD8+T cells with K562 target cells expressing variable levels of HER2 antigen (Hernandez-Lopez 2021). Target cell lysis and expression of the T cell activation marker CD25 were measured after 72 hours of coculture (FIG. 3C).

Using the CD19BBz CAR, it was first demonstrate that incubation of 1 μM of MLN4924 for 5 hours at 37 C can rescue STUD knockdown of CAR expression (FIG. 3D). Engineered CD8+T cells expressing were cocultured with NALM6 target cells and measured activation of the CAR by quantifying target cell lysis and expression of the T cell activation marker CD25. From these experiments, greatly diminished cell lysis and expression of CD25 from cells expressing the memSTUD relative to cells expressing a CAR alone or the non-functional STUD (FIG. 3C&D) was observed. From these data, it was concluded that membrane tethering of a STUD is necessary for sufficient knockdown of CAR proteins and that this knockdown is able to functionally disable the CAR.

### Example 9

Design of New Synthetic Receptor Allows for Antigen Triggered Degradation of Cytosolic Proteins

Synthetic Notch receptors, and the newly published SyNthetic Intramembrane Proteolysis Receptors (SNIPRs), are a class of synthetic proteins that borrow from the Notch family of receptors (Morsut, et al Cell 1016 164: 780-⁷91, Zhu et al bioRxiv, posted May 23, 2021, Roybal, et al Cell 2016 167, 419-432) These molecules have a customizable intracellular transcription factor that gets released from the membrane upon antigen recognition and binding. It was hypothesized that we could exchange the transcription factor for a STUD to result in antigen-dependent degradation of a cytosolic target. By combining the extracellular antigen recognition domain and the transmembrane and juxtairembrane domains from SNIPRs, a novel proteolytic receptor, the ‘NotchSTUD’, was designed.

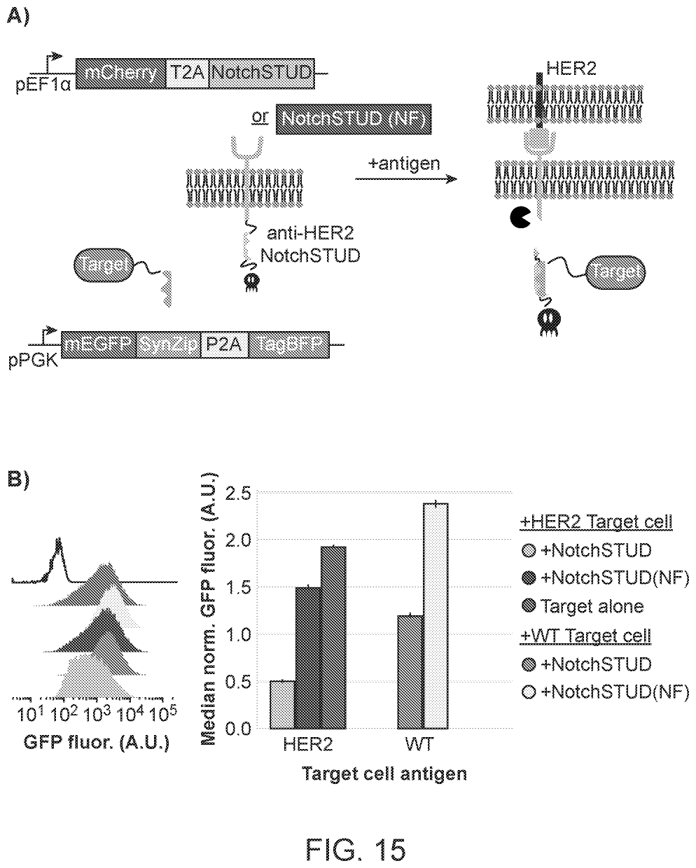

Here, the NotchSTUD was used to degrade a GFP-SynZip target in an antigen-dependent manner. CD4+primary human T cells were lentivirally induced with a two plasmid system. The first encodes the NotchSTUD and muCherry cotransduction marker and the second encodes the same GFP target described in FIG. 12. Following lentiviral transduction, cells expressing both of these plasmids by FACS were isolated and the cells were cocultured with K562 target cells expressing either the NotchSTUD antigen (HER2) or wild-type cells that express no antigen (FIG. 14A). After 72 hours of culture, we measure GFP fluorescence by flow cytometry. GFP fluorescence was quantified by normalizing using the same method as described above. From these assays, we observe modest decrease in the median normalized GFP fluorescence in cells that express the NotchSTUD co-cultured with HER2 target cells relative to the same cells cocultured with WT target cells (FIG. 15B). Minimal change in normalized GFP fluorescence was observed in cells that express a nonfunctional NotchSTUD.

### Example 10

STUDs can be Composed into Negative Feedback Circuit to Regulate Synthetic Transcription Factor (SynTF).

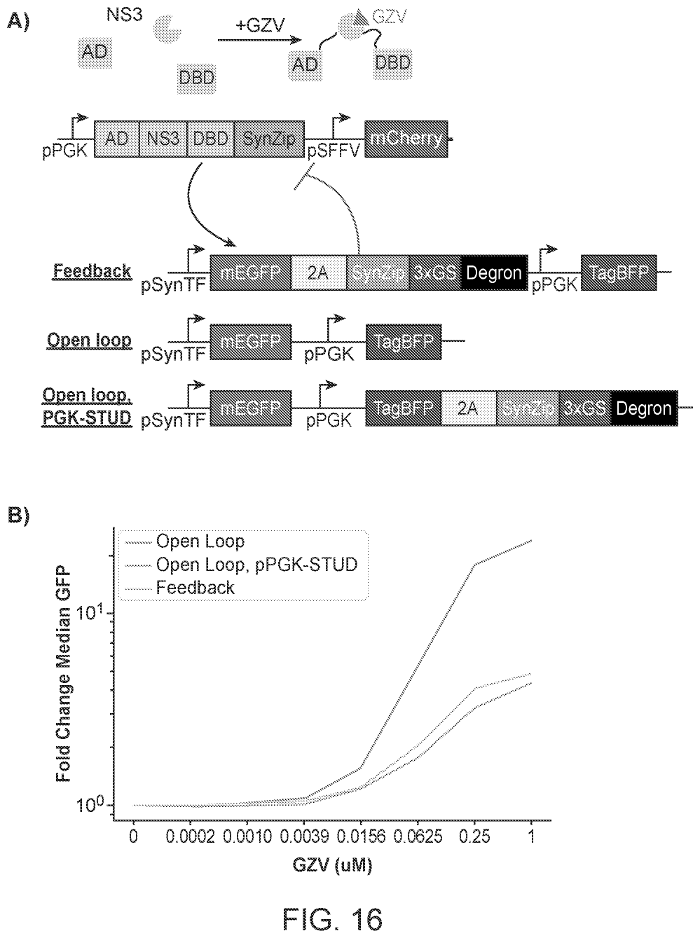

It is shown that STUDs can be composed into molecular circuits by demonstrating the use of STUDs in a negative feedback loop. The circuit has three components: (1) a synthetic drug-inducible transcription factor (SynTF) fused to a SynZip, (2) a GFP reporter, and (3) a SynZip STUD that targets the SynTF (FIG. 16A). The SynTF relies on previously published NS3 protease from the Hepatitis C virus and the small-molecule drug grazoprevir (GZV) (cite). GZV is an inhibitor of the NS3 protease and in the absence of GZV, the protease is active and the SynTF is non-functional. On the other hand, in the presence of GZV, the NS3 is inhibited and the SynTF is stable and functional. A stable SynTF then drives the production of the GFP reporter and the STUD feedback cassette. We also built two open loop control circuits. The first has SynTF drive the GFP reporter alone while the second has a constitutively expressed STUD that continuously degrades the SynTF. We introduce component 1 and components 2 and 3 as a two plasmid system into Jurkat T cells by lentivirally transduction. 72 hours after removal of lentivirus, we induce these cells with GZV for 72 hours at 37 C. After incubation, we assay for GFP fluorescence by flow cytometry and gate on cells expressing the co-transduction markers (mCherry and tagBFP) for both plasmids relative to a untransduced control. Looking at the fold change of the median GFP at each concentration of drug relative to a DMSO vehicle control, we find that the STUD negative feedback circuit inhibits the SynTF closely resembling the inhibition of the constitutive STUD open loop control (FIG. 16B). From these data, it was concluded that the STUD can be incorporated into negative feedback loops and powerfully regulate synTFs.

### Example 11

Dose Dependent Degradation of STUDs Allow for Use as ON Switch CAR.

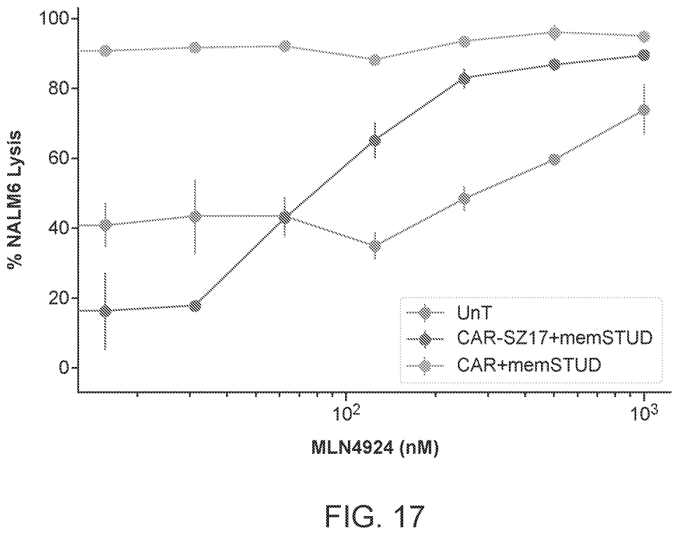

CD8+primary human T cells were engineered with an antiCD19BBz CAR fused to a SynZip and a memSTUD that binds the SynZip using the same plasmids outlined in FIG. 14A by lentiviral transduction. As a control, we also lentivirally transduce CD8+primary human T cells with an antiCD19BBz and a SynZip memSTUD which cannot bind the CAR. These engineered cells and an untransduced control were cocultured with NALM6 target cells for 72 hours at 37 C in the presence of MLN4924 ranging from 1 μM to 0.015625 μM and a DMSO vehicle control. After coculture, target cell lysis was assayed by flow cytometry. Specific target cell lysis of each cell line relative to NALM6 cells cultured alone in the same conditions was then calculated. It was found that that the activity of the STUD can be titrated such that lysis by CAR is dose dependent which can be used as an ON switch CAR in future applications (FIG. 17).

Materials and Methods

Cytosolic STUD for targeting GFP: Cytosolic STUDs were introduced by lentiviral transduction of two plasmids. The first encodes a green fluorescent protein (GFP) which will be a target for degradation alongside a BFP as a co-transduction marker. The second encodes the STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of GFP fluorescence.

Using proteasome inhibitor to explore cytosolic GFP mechanism: To ascertain the mechanism by which the STUD degrades cytosolic GFP, we incubated cells with 5 μM of the proteasome inhibitor MG132 for 1 and 3 hours. Cells were then washed with PBS and analyzed by flow cytometry. Using the same 2-plasmid system as described above, we measured changes in GFP fluorescence relative to controls.

Membrane targeting STUD: Membrane targeting cells were introduced by lentiviral transduction of two plasmids. The first encodes a chimeric antigen receptor (CAR) or synthetic Notch (SynNotch) protein which will be a target for degradation alongside a BFP as a co-transduction marker. The second encodes the membrane localized STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of CAR/SynNotch. CAR and SynNotch expression was measured by antibody staining for a peptide tag fused to the extracellular domain of the CAR/SynNotch.

Cell culture for Lenti-X 293T cells: Lenti-X 293T packaging cells (Clontech #1113ID) were cultured in medium consisting of Dulbecco's Modified Eagle Medium (DMEM) (Gibco #10569-010) and 10% fetal bovine serum (FBS) (University of California, San Francisco [UCSF] Cell Culture Facility). Lenti-X 293T cells were cultured in T150 or T225 flasks (Corning #430825 and #431082) and passaged every 2-3 days upon reaching 70-80% confluency. To passage, cells were treated with TrypLE express (Gibco #12605010) at 37 C for 5 minutes. Then, 10 mL of media was used to quench the reaction and cells were collected into a 50 mL conical tube and pelleted by centrifugation (400×g for 4 minutes). Cells were cultured until passage 30 whereupon fresh Lenti-X 293 T cells were thawed.

Cell culture for HEK 293T cells: HEK 293T cells (UCSF Cell Culture Facility) were cultured in medium consisting of Dulbecco's Modified Eagle Medium (DMEM) (Gibco #10569-010) and 10% fetal bovine serum (FBS) (UCSF Cell Culture Facility). HEK 293T cells were cultured in T75 flasks (Coming #430641U) and passaged every 2-3 days upon reaching 70-80% confluency.

Cell culture for 3T3 cells: 3T3 cells were cultured in medium consisting of Dulbecco's Modified Eagle Medium (DMEM) (Gibco #10569-010) and 10% fetal bovine serum (FBS) (UCSF Cell Culture Facility). 3T3 cells were passaged upon reaching 70-80% confluency. To pass, cells were treated with TrypLE express at 37 C for 3 minutes. Then, 10 mL of media was added to quench the reaction and cells were collected into a 50 mL conical tube and pelleted by centrifugation (400×g for 4 minutes). Pellet was resuspended in 5 mL and 1 mL of resuspended pellet was added to a T25 flask (Corning #430639) containing 10 mL of media.

Cell culture for Jurkat T cells: Jurkat T cells (UCSF Cell Culture Facility) were cultured in media consisting of RPMI-1640 (ThermoFisher Scientific #11875093), 10% FBS (UCSF Cell Culture Facility) and 1% antibiotics-antimycotics (ThermoFisher Scientific #15240062). To passage, cells were maintained at a concentration of 1×10∧6 cells/mL in a T150 flask. Cells were cultured until passage 30 whereupon fresh Jurkat T cells were thawed.

Cell culture for K562 myelogenous leukemia cells: K562 cells were cultured in media consisting of Iscove's Modified Dulbecco's Medium (ThermoFisher Scientific #12440053), 10% FBS (UCSF Cell Culture Facility) and 1% Gentamicin (ThermoFisher Scientific #15750078). To passage, cells were maintained at a concentration of 1×10∧6 cells/mL in a T25 flask.

Culture of mouse embryonic stem cells (mESCs): mESCs were cultured in “Serum Free ES” (SFES) media supplemented with 2i. SFES media consists of 500 mL DMEM/F12 (Gibco #11320-033), 500 mL Neurobasal (Gibco #21103-049), 5 mL N2 Supplement (Gibco #17502-048), 10 mL B27 with retinoic acid (gibco #17504-044), 6.66 mL 7.5% BSA (Gibco #15260-037), 10 mL 100× GlutaMax (Gibco #35050-061), and 10 mL 100× Pen/Strep. To make “2i SFES”, 1 nM PD03259010 (Selleckchem #S1036), 3 nM CHIR99021 (Selleckchem #S2924) and 1000 units/mL LIF (ESGRO #ESG1106) were added to 45 mL SFES. Prior to use, 1-thioglycerol (MTG; Sigma M6145) was diluted 1.26% in SFES and added 1:1000 to 2i SFES media. To passage, mESCs were treated with 1 mL of accutase in a 6 well plate (Coming #353046) for 5 minutes at room temperature (RT). After incubation, cells were mixed by pipette and moved to a 15 mL conical tube, supplemented with 10 mL SFES and spun at 300×g for 3 minutes. Then, media was removed and cells were counted using the Countess II Cell Counter (ThermoFisher) according to the manufacturer's instructions. Cells were then plated in 6 well plates that had gelatinized with 1% gelatin for 30 minutes at 37 C at 5×10∧5 cells per well in 2 mL of 2i SFES. Media was changed every day and cells were split every other day.

Primary Human T Cell Isolation and Culture: Primary CD4+ and CD8+T cells were isolated from anonymous donor blood after apheresis by negative selection (STEMCELL Technologies #15062 and 15023). T cells were cryopreserved in RPMI-1640 (Corning #10-040-CV) with 20% human AB serum (Valley Biomedical, #HP1022) and 5% DMSO (Sigma-Aldrich #472301). After thawing, T cells were cultured in human T cell medium (hTCM) consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

Lentiviral transduction of primary T cells: Pantropic VSV-G pseudotyped lentivirus was produced via transfection of Lenti-X 293T cells with a modified pHR′ SIN:CSW transgene expression vector and the viral packaging plasmids pCMVdR8.91 and pMD2.G using Fugene HD (Promega #E2312). Primary T cells were thawed the same day, and after 24 hr in culture, were stimulated with Dynabeads Human T-Activator CD3/CD28 (Thermo Scientific #1113ID) at a 1:3 cell:bead ratio. At 48 hr, viral supernatant was harvested and concentrated using the Lenti-X concentrator (Takara, #631231) according to the manufacturer's instructions. Briefly, viral supernatant was harvested and potential contaminants were filtered using a 0.45 μM filter (Millipore Sigma #SLHV033RS). Lenti-X concentrator solution was added at a 1:3 viral supernatant:concentrator ratio, mixed by inversion, and incubated at 4 C for at least 2 hours. Supernatant-concentrator mix was pelleted by centrifugation at 1500×g at 4 C for 45 minutes, supernatant was removed and pellet was resuspended using 100 μL media or PBS (UCSF Cell Culture Facility) for each well of T cells. Typically, 2 wells of a 6 well plate was concentrated for 1 well of a 24 well plate plated with 1 million T cells on day of transfection. The primary T cells were exposed to the virus for 24 hr and viral supernatant was exchanged for fresh hTCM supplemented with IL-2 as described above. At day 5 post T cell stimulation, Dynabeads were removed and the T cells expanded until day 12-14 when they were rested for use in assays. For co-culture assays, T cells were sorted using a Sony SH-800 cell sorter on day 5-6 post stimulation.

Construct assembly: All plasmids were constructed using a previously described hierarchical DNA assembly method based on Golden Gate cloning(Lee 2015, Fonseca 2019). Plasmids were verified by sequencing and/or restriction digest and gel electrophoresis.

Flow cytometry: All flow cytometry data was obtained using a LSR Fortessa (BD Biosciences). All assays were run in a 96-well round bottom plate (Fisher Scientific #08-772-2C). Samples were prepared by pelleting cells in the plate using centrifugation at 400×g for 4 minutes. Supernatant was then removed and 200 μL of PBS (UCSF Cell Culture facility) was used to wash cells. The cells were again pelleted as described above and supernatant was removed. Cells were resuspended in 120 μL of Flow buffer (PBS+2% FBS) and mixed by pipetting prior to flow cytometry assay.

Inhibitor Assays: 100,000 cells were plated in a 96 well round bottom plate with either 5 μM MG-132 (Sigma-Aldrich #M7449-200UL), 1 μM MLN4924(Active Biochem #A-1139), 100 nM Bafilomycin A1(Enzo Life Sciences #BML-CM110-0100), or DMSO vehicle control and incubated at 37 C for 5 hours. After incubation, cells were pelleted by centrifugation at 400×g for 4 minutes. Supernatant was then removed and cells were washed once with 200 μL PBS. Cells were pelleted again (400×g for 4 minutes) and resuspended in flow buffer (PBS+2% FBS) for assay by flow cytometry.

Antibody staining: All experiments using antibody staining were performed in 96 well round bottom plates. Cells for these assays were pelleted by centrifugation (400×g for 4 minutes) and supernatant was removed. Cells were washed once with 200 μL of PBS and pelleted again by centrifugation (400×g for 4 minutes) and the supernatant was removed. Cells were resuspended in a staining solution of 50 μL PBS containing fluorescent antibody stains of interest. Anti-myc antibodies (Cell Signaling Technologies #2233S and #2279S) was used at a 1:100 ratio while antiV5 (ThermoFisher Scientific #12-679642) and antiFLAG (R&D Systems #IC8529G-100) antibodies were used at a 1:50 ratio for flow cytometry assays. For FACS, all antibodies were used in a 1:50 ratio in 100 uL.

Generation of coculture target cells: HER2-expressing K562 target cells were previously characterized in the literature and were a gift from Dr. Wendell Lim (Hernandez-Lopez 2021). CD19-expressing K562 cells were generated by lentiviral transduction and antibiotic selection with 2 ug/mL puromycin for one week.

NALM6 cell culture: NALM6 cells were cultured in medium consisting of RPMI-1640, 10% fetal bovine serum (FBS) (University of California, San Francisco [UCSF] Cell Culture Facility), and 1% antibiotics-antimycotics. To passage, cells were maintained at a concentration of 1×10∧6 cells/mL in a T25 flask.

Co-culture assays: For all assays, T cells and target cells were co-cultured at a 1:1 ratio with cell numbers varying per assay. All assays contained between 10,000 and 50,000 of each cell type. The Countess II Cell Counter (ThermoFisher) was used to determine cell counts for all assays set up. T cells and target cells were mixed in 96-well round bottom tissue culture plates in 200 μL T cell media, and then plates were centrifuged for 1 min at 400×g to initiate interaction of the cells prior to incubation at 37 C.

Data analysis: Data analysis was performed using the FlowJo software (FlowJo LLC.) and Python. For co-culture assays, desired cell populations were isolated by FACS using a Sony SH800 cell sorter. For non co-culture assays, desired cell populations were isolated by gating in FlowJo following flow cytometry.

Grazoprevir (GZV) induction: 25,000 Jurkat T cells were seeded into 96 well round bottom plates in 100 μL fresh media. 100 μL containing media containing a 2×concentration of GZV was added to each well of seeded cells. Cells with GZV were incubated at 37 C for 72 hours. DMSO vehicle at the same concentration as the max GZV concentration was added to cells as a control.

MLN dose response: 25,000 CD8+primary human T cells were seeded into 96 well round bottom plates in 100 μL fresh media. 100 μL containing media containing a 2×concentration of MLN4924 was added to each well of seeded cells. Cells with MLN4924 were incubated at 37 C for 72 hours. DMSO vehicle at the same concentration as the max MLN4924 concentration was added to cells as a control.

### REFERENCES

1. Bonger, K. M., Chen, L.-C., Liu, C. W. & Wandless, T. J. Small-molecule displacement of a cryptic degron causes conditional protein degradation. Nat. Chem. Biol. 7, 531-537 (2011).
2. Daniel, K. et al. Conditional control of fluorescent protein degradation by an auxin-dependent nanobody. Nat. Commun. 9, 3297 (2018).
3. Arai, R., Ueda, H., Kitayama, A., Kamiya, N. & Nagamune, T. Design of the linkers which effectively separate domains of a bifunctional fusion protein. Protein Eng. 14, 529-532 (2001).
4. Wu, C. Y., Roybal, K. T., Puchner, E. M. & Onuffer, J. Remote control of therapeutic T cells through a small molecule-gated chimeric receptor. Science (2015).

1. A therapeutic cell that expresses a fusion protein comprising:
(a) a target-binding domain; and
(b) a degradation domain that is heterologous to the target-binding domain, wherein the degradation domain is a degron or E3 ligase-recruiting domain,
wherein, in the therapeutic cell, binding of the fusion protein to a target protein via the target-binding domain induces degradation of the target protein.

2. The therapeutic cell of claim 1, wherein the fusion protein further comprises a transmembrane domain and wherein, in the therapeutic cell, the target-binding domain and degradation domain are intracellular and binding of the fusion protein to a transmembrane protein via the target-binding domain induces degradation of the transmembrane protein.

3. The therapeutic cell of claim 1, wherein the fusion protein further comprises (c), a linker, between the target-binding domain of (a) and the degradation domain of (b).

4. The therapeutic cell of claim 1, wherein the degradation domain is a degron.

5. The therapeutic cell of claim 4, wherein the degron is a C-terminal RRRG (SEQ ID NO:32) sequence.

6. The therapeutic cell of claim 1, wherein the degradation domain is an E3 ligase-recruiting domain.

7. The therapeutic cell of claim 6, wherein there are no lysines on the surface of the E3 ligase-recruiting domain and/or the target binding domain.

8. The therapeutic cell of claim 6, wherein the E3 ligase-recruiting domain directly binds to an E3 ligase.

9-12. (canceled)

13. The therapeutic cell of claim 1, wherein the target-binding domain is a scFv or nanobody.

14. The therapeutic cell of claim 1, wherein the target-binding domain is a non-antibody target-binding domain.

15. The therapeutic cell of claim 1, wherein the target-binding domain is a synthetic leucine zipper.

16. The therapeutic cell of claim 13, wherein the target-binding domain binds to a motif having a post-translational modification.

17. The therapeutic cell of claim 1, wherein the target protein is endogenous to the cell.

18. The therapeutic cell of claim 1, wherein the target protein is exogenous to the cell.

19. The therapeutic cell of claim 18, wherein the target protein comprises a synthetic leucine zipper and binding between the fusion protein and the target protein is via leucine zippers.

20. The therapeutic cell of claim 18, wherein binding of the fusion protein to the target protein is chemically inducible.

21. The therapeutic cell of claim 1, wherein the cell is an immune cell.

22. The therapeutic cell of claim 21, wherein the immune cell is immunostimulatory.

23. The therapeutic cell of claim 22, wherein the immune cell is a chimeric antigen receptor T cell (CAR-T).

24. The therapeutic cell of claim 23, wherein the target is the CAR, or component of signal transduction pathway activated the CAR.

25-28. (canceled)

29. A method comprising:
incubating a cell of any prior claim, thereby degrading the target protein.

30. (canceled)

31. The method of claim 29, further comprising inhibiting degradation of the target protein by a proteasome inhibitor.

32. A method comprising: administering a cell of claim 1 to a patient in need thereof.

33-35. (canceled)


SEQUENCE LISTING (50 sequences)

SEQ ID NO: 1 - protein, 23 aa, Artificial sequence
  synthetic sequence
RGKRGGHATNYRIVAPRSRDERG

SEQ ID NO: 2 - protein, 23 aa, Artificial sequence
  synthetic sequence
ESLSAIEAELEKVAHQLQALRRG

SEQ ID NO: 3 - protein, 23 aa, Artificial sequence
  synthetic sequence
KISGGPHISYPPLHEWVLREGEE

SEQ ID NO: 4 - protein, 23 aa, Artificial sequence
  synthetic sequence
KISGGPHISYPPLHEWVLREGAA

SEQ ID NO: 5 - protein, 24 aa, Artificial sequence
  synthetic sequence
LRELDLAQEKTGWFALGSRSHGTL

SEQ ID NO: 6 - protein, 24 aa, Artificial sequence
  synthetic sequence
LRELDLAQEKTGWFALGSRHCGRT

SEQ ID NO: 7 - protein, 23 aa, Artificial sequence
  synthetic sequence
YKKAGSGIPLRMNSLFRKRNKGK

SEQ ID NO: 8 - protein, 23 aa, Artificial sequence
  synthetic sequence
VFSDLKNESGQEDKKRLLLGLDR

SEQ ID NO: 9 - protein, 22 aa, Artificial sequence
  synthetic sequence
VFSDLKNESGQEDKKRLLLGLD

SEQ ID NO: 10 - protein, 23 aa, Artificial sequence
  synthetic sequence
DGEDEGYFQELLGSVNSLLKELR

SEQ ID NO: 11 - protein, 22 aa, Artificial sequence
  synthetic sequence
DGEDEGYFQELLGSVNSLLKEL

SEQ ID NO: 12 - protein, 23 aa, Artificial sequence
  synthetic sequence
LEKEKLQARQQQLLKKIEHARTC

SEQ ID NO: 13 - protein, 20 aa, Artificial sequence
  synthetic sequence
LEKEKLQARQQQLLKKIEHA

SEQ ID NO: 14 - protein, 23 aa, Artificial sequence
  synthetic sequence
GPGPNGQRFCINSAALKFKPRKH

SEQ ID NO: 15 - protein, 23 aa, Artificial sequence
  synthetic sequence
LRNAQMKNAIRKLCSRKAISSVK

SEQ ID NO: 16 - protein, 23 aa, Artificial sequence
  synthetic sequence
LRNAQMKNAIRKLCSRKAISSDK

SEQ ID NO: 17 - protein, 23 aa, Artificial sequence
  synthetic sequence
RRSCNDCQQMIVKLGGGTAIAAS

SEQ ID NO: 18 - protein, 23 aa, Artificial sequence
  synthetic sequence
RRSCNDCQQMIVKLGGGTAIADS

SEQ ID NO: 19 - protein, 23 aa, Artificial sequence
  synthetic sequence
QKSRKVDSKSLFHYRQYSAGKAA

SEQ ID NO: 20 - protein, 23 aa, Artificial sequence
  synthetic sequence
QKSRKVDSKSLFHYRQYSAGKDD

SEQ ID NO: 21 - protein, 28 aa, Artificial sequence
  synthetic sequence
QKSRKVDSKSLFHYRQYSAGKAAKASTN

SEQ ID NO: 22 - protein, 23 aa, Artificial sequence
  synthetic sequence
REIQGIFFKEDSHKESNDCSCGG

SEQ ID NO: 23 - protein, 23 aa, Artificial sequence
  synthetic sequence
SLTDVALEHHEECDCVCRGSTGG

SEQ ID NO: 24 - protein, 23 aa, Artificial sequence
  synthetic sequence
RRLDGKEEDEKMSRASDRFRGLR

SEQ ID NO: 25 - protein, 19 aa, Artificial sequence
  synthetic sequence
TRGVEEVAEGVVLLRRRGN

SEQ ID NO: 26 - protein, 10 aa, Artificial sequence
  synthetic sequence
GIPLRNLGIR

SEQ ID NO: 27 - protein, 15 aa, Artificial sequence
  synthetic sequence
GIPLRQRKLQRTSRG

SEQ ID NO: 28 - protein, 15 aa, Artificial sequence
  synthetic sequence
GIPLRQRKLQRTSRA

SEQ ID NO: 29 - protein, 17 aa, Artificial sequence
  synthetic sequence
GIPLRPHKRLLKGSQYG

SEQ ID NO: 30 - protein, 18 aa, Artificial sequence
  synthetic sequence
TRGVEEVAEGVVLLRRRG

SEQ ID NO: 31 - protein, 6 aa, Artificial sequence
  synthetic sequence
TRRRGN

SEQ ID NO: 32 - protein, 4 aa, Artificial sequence
  synthetic sequence
RRRG

SEQ ID NO: 33 - protein, 115 aa, Artificial sequence
  synthetic sequence
QIFVKTLTGKTITLEVESSDTIDNVKSKIQDKEGIPPDQQRLIFAGKQLEDGRTLSDYNIQKESTLHLVLRLRGGRHGSGAWLLPVSLVRRRTTLAPNTQTASPRALADSLMQRS

SEQ ID NO: 34 - protein, 21 aa, Artificial sequence
  synthetic sequence
DDLLLPQDVEEFFEGPSEALR

SEQ ID NO: 35 - protein, 7 aa, Unknown
  HIF-1a
ALAPYIP

SEQ ID NO: 36 - protein, 14 aa, Unknown
  RNaseA-hsc70-hemoglobin
KFERQKILDQRFFE

SEQ ID NO: 37 - protein, 21 aa, Unknown
  p54(92-112)
PLSSSVPSQKTYQGSYGFRLG

SEQ ID NO: 38 - protein, 4 aa, Unknown
  p54(92-112)
GSYG

SEQ ID NO: 39 - protein, 10 aa, Unknown
  SRC3
DVQKADVSST

SEQ ID NO: 40 - protein, 8 aa, Unknown
  Nanog
SPDSSTSP

SEQ ID NO: 41 - protein, 5 aa, Unknown
  iNOS
DINNN

SEQ ID NO: 42 - protein, 5 aa, Artificial sequence
  synthetic sequence
GSGGS

SEQ ID NO: 43 - protein, 4 aa, Artificial sequence
  synthetic sequence
GGGS

SEQ ID NO: 44 - protein, 4 aa, Artificial sequence
  synthetic sequence
GGSG

SEQ ID NO: 45 - protein, 5 aa, Artificial sequence
  synthetic sequence
GGSGG

SEQ ID NO: 46 - protein, 5 aa, Artificial sequence
  synthetic sequence
GSGSG

SEQ ID NO: 47 - protein, 5 aa, Artificial sequence
  synthetic sequence
GSGGG

SEQ ID NO: 48 - protein, 5 aa, Artificial sequence
  synthetic sequence
GGGSG

SEQ ID NO: 49 - protein, 5 aa, Artificial sequence
  synthetic sequence
GSSSG

SEQ ID NO: 50 - protein, 4 aa, Artificial sequence
  synthetic sequence
TRGN